(19) United States
(12) Patent Application Publication
(10) Pub. No.: US 2008/0108581 A1
(43) Pub. Date: May 8, 2008

(54) Compounds and Their Use for Specific and Simultaneous Inhibition of Genes Involved In Diseases and Related Drugs

(75) Inventors: Paola Barbara Arimondo, Paris (FR); Alexandre Boutorine, Sainte Geneviève des Bois (FR); Jian-Sheng Sun, Saint Maur des Fossès (FR); Christian Bailly, Wasquehal (FR); Claude Helene, Paris (FR); Therese Garestier, Paris (FR)

Correspondence Address: NIXON & VANDERHYE, PC, 901 NORTH GLEBE ROAD, 11TH FLOOR, ARLINGTON, VA, 22203, US

(73) Assignees: INSTITUT NATIONAL DE LA SANTE ET DE LA RECHERCHE MEDICAL (I.N.S.E.R.M.), Paris, Cedex (FR); CENTRE NATIONAL DE LA RECHERCHE SCIENTIFIQUE (C.N.R.S.), Paris, Cedex (FR); MUSEUM NATIONAL D'HISTOIRE NATURELLE, Paris, Cedex (FR)

(21) Appl. No.: 10/549,129

(22) PCT Filed: Mar. 18, 2004
(86) PCT No.: PCT/EP04/04022

§ 371 Date: Jul. 24, 2007

(30) Foreign Application Priority Data
Mar. 18, 2003 (FR) FR 03 03 311

(51) Int. Cl. A61K 48/00; A61K 31/4162; A61P 43/00

(52) U.S. Cl. 514/44; 514/283

(57) ABSTRACT

The invention relate to the use of a compound of formula A-B—C Wherein A is a DNA sequence-specific ligand capable of simultaneously and specifically recognizing a sequence common to genes of pathological interest; B is a linker arm, said linker arm being bound to the 3′ end of A; C is a topoisomerase I posion; for the preparation of a drug for the treatment of a disease brought about by the expression of a gene and said gene is inhibited by the stabilized topoisomerase I-mediated DNA cleavage. Application, particularly, for the treatment of infective microorganism or virus, dismetabolic disease and autoimmune disease.

The invention relates to products, processes for their preparation, methods for their use and compositions containing them which make it possible to simultaneously inhibit the expression of several genes involved in a pathology by inducing irreversible lesions on these genes. It more particularly relates to a method and products that selectively target a chosen sequence and that inhibit simultaneously a common sequence shared by several genes concern to a given pathology.

Triple helix-forming oligonucleotides (TFOs) were developed in the Biophysics Laboratory of the Muséum National d'Histoire Naturelle USM 0503 Unit INSERM UR565, CNRS UMR 5153, with the aim of interfering specifically with the expression of certain genes. These TFOs have been used for other applications, for example the purification of plasmids or the chemical modification of the target sequence. In 1997, an in vitro study showed that the chemical coupling of a derivative of camptothecin, a topoisomerase I inhibitor or, more exactly, poison, to a triple helix forming oligonucleotide directs the cleavage of the DNA by topoisomerase I specifically to the oligopyrimidine-oligopurine sequence targeted by the triple helix oligonucleotide (Matteucci et al. J. Am. Chem. Soc. 119 (1997) pp 6939-6940).

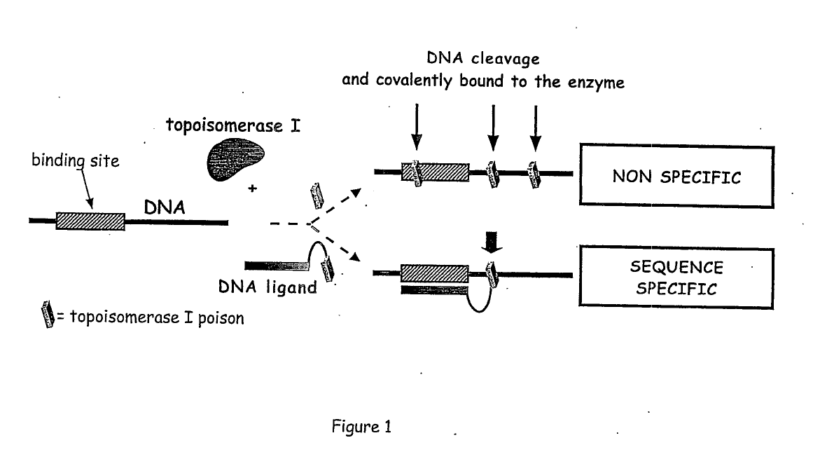

As already described in the literature and in particular in the publications of the inventors (Arimondo et al. 1999, 2000, 2001a,b, 2002), topoisomerase I inhibitors coupled to a specific DNA ligand become specific to the binding site of the DNA ligand. In the context of the present invention, the product topoisomerase I poison attached covalently to the DNA ligand is also called hereafter conjugate. This approach makes it possible to develop antitumoral agents, the mechanism of action of which is based on the selective modulation of a single gene, involved in the tumoral state (FIG. 1). Certain topoisomerase I inhibitors, such as two derivatives of camptothecin (CPT in short), are used in clinical practice, but have considerable toxicity levels, potentially correlated to their low sequence specificity.

The problem of the selectivity of antitumor drugs is also present in other type of chemotherapeutical drugs, such as antibiotics.

Targeting of drugs can be seen as a general problem in modern therapy and involves also dismetabolic and autoimmune diseases.

It has now been found that specific conjugates comprising a topoisomerase I poison and a DNA sequence-specific ligand, connected by a linker arm, are capable of directing the action of the topoisomerase I poison specifically on a gene of interest, the expression of which is related with a disease, in particular a tumor or an infective disease.

The problems and drawbacks referred to in the prior art are overcome according to the invention, the main subjects of which are the following.

The present invention first of all relates to the use of a compound of formula

A-B—C

wherein

### A is a DNA sequence-specific ligand capable of simultaneously and specifically recognizing a sequence common to the genes of pathological interest;

### B is a linker arm, said linker arm being bound to the 3′ end of A;

### C is a topoisomerase I poison; for the preparation of a medicament for the treatment of a disease brought about by the expression of genes and said genes are inhibited by the stabilized topoisomerase I-mediated DNA cleavage.

In the development of the present invention, the present inventors have also found new compounds of formula A-B—C, which are a specific object of the present invention.

The present invention also relates to processes for the preparation of the above compounds, compositions comprising them and methods of using said compounds in the development of new drugs and in pharmacological tests.

A further object of the present invention is a method for simultaneously inhibiting the expression of several target genes coding for proteins of pathological interest, in particular involved in the development and maintenance of tumors, or viral and pathogenic proteins, or proteins involved in dismetabolic or autoimmune proteins comprising the steps of:
(i) directing the action of at least one topoisomerase I inhibitor towards a site specific to said genes by said conjugate, at least one topoisomerase inhibitor to at least one DNA sequence-specific ligand capable of simultaneously and specifically recognizing a sequence common to said target genes,
(ii) recognition by the said ligand of the said conjugate of the said genes in the genome and obtaining the binding of said ligand to said targets,
(iii) induction of topoisomerase I-mediated DNA cleavage, and inhibiting the expression of the said genes.

According to the invention, this method can be carried out in particular in vitro and in vivo.

By using said arrangements, it is possible to direct the effect of the topoisomerase I inhibitor(s) to the DNA-specific sites and to selectively induce a break at these sites by the topoisomerase I. The inhibitor(s) coupled to the DNA-specific ligand becomes (become) itself (themselves) specific of the DNA ligand fixation site. Advantageously, the targeted DNA sequences can be selected depending on the kind of the pathology.

According to a preferred embodiment of the invention, said genes are selected among those the expression of which controls the development and maintenance of tumoral state of the cells. In a particularly preferred embodiment, the genes are selected from the group consisting of IGF-1, IGF-1R, VEGF, BCL2.

According to another preferred embodiment of the invention, said genes are selected among those of an infective micro-organism or a virus. In a particularly preferred embodiment, the genes are those of a pathogen selected from the group consisting of HIV or HCV virus.

According to a still further preferred embodiment of the invention, said genes are selected among those involved in a dismetabolic disease.

According to a still further preferred embodiment of the invention, said genes are selected among those involved in an autoimmune disease.

According to the invention, the topoisomerase I inhibitor or more precisely poison, is a molecule that stabilize the DNA/topo I cleavage complex mediated by the catalytic action of topoisomerase I. The poison is advantageously selected from the group consisting of intercalating agents, such as indolocarbazoles and derivatives thereof, indenoisoquinolines, non-intercalating agents, such as camptothecin and derivatives thereof, minor groove ligands, such as the benzimidazoles and derivatives thereof.

According to a preferred embodiment of the present invention, the poison is camptothecin, more preferably a camptothecin derivative.

A preferred camptothecin derivative is a compound of formula (I)

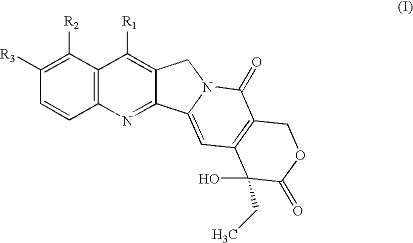

wherein:

R1 is a —C(R₅)═N—(O)ₙ—R₄ group, in which n is the number 0 or 1, R₄ is hydrogen or a straight or branched C₁-C₈ alkyl or C₂-C₈ alkenyl group, or a C₃-C₁₀ cycloalkyl group, or a straight or branched (C₃-C₁₀) cycloalkyl-(C₁-C₈) alkyl group, or a C₆-C₁₄ aryl group, or a straight or branched (C₆-C₁₄) aryl-(C₁-C₈) alkyl group, or a heterocyclic group or a straight or branched heterocyclo-(C₁-C₈) alkyl group, said heterocyclic group containing at least one heteroatom selected from an atom of nitrogen, optionally substituted with a (C₁-C₈) alkyl group, and/or an atom of oxygen and/or of sulphur; said alkyl, alkenyl, cycloalkyl, cycloalkylalkyl, aryl, arylalkyl, heterocyclic or heterocyclo-alkyl groups may optionally be substituted with one or more groups selected from: halogen, hydroxy, keto, C₁-C₈ alkyl, C₁-C₈ alkoxy, phenyl, cyano, nitro, —NR₆R₇, where R₆ and R₇, which may be the same or different, are hydrogen, straight or branched (C₁-C₈) alkyl, the —COOH group or one of its pharmaceutically acceptable esters; or the —CONR₈R₉ group, where R₈ and R₉, which may be the same or different, are hydrogen, straight or branched (C₁-C₈) alkyl, phenyl; or R₄ is a (C₆-C₁₀) aryl or (C₆-C₁₀) arylsulphonyl residue, optionally substituted with one or more groups selected from the group consisting or: halogen, hydroxy, straight or branched C₁-C₈ alkyl, straight or branched C₁-C₈ alkoxy, phenyl, cyano, nitro, —NR₁₀R₁₁, where R₁₀ and R₁₁, which may be the same or different, are hydrogen, straight or branched C₁-C₈ alkyl; or R₄ is a polyaminoalkyl residue, in particular —(CH₂)ₘ—NR₁₂—(CH₂)ₚ—NR₁₃—(CH₂)_{q}—NH₂, wherein m and p are an integer from 2 to 6 and q is an integer from 0 to 6, extremes included and R₁₂ and R₁₃ are a straight or branched C₁-C₈ alkyl group, for example N-(4-aminobutyl)-2-aminoethyl, N-(3-aminopropyl)-4-aminobutyl, N-[N-3-aminopropyl)-N-(4-aminobutyl)]-3-aminopropyl; or R₄ is a glycosyl residue, for example 6-D-galactosyl or 6-D-glucosyl; R₅ is hydrogen, straight or branched C₁-C₈ alkyl, straight or branched C₂-C₈ alkenyl, C₃-C₁₀ cycloalkyl, straight or branched (C₃-C₁₀) cycloalkyl-(C₁-C₈) alkyl, C₆-C₁₄ aryl, straight or branched (C₆-C₁₄) aryl-(C₁-C₈) alkyl; R₂ and R₃, which may be the same or different, are hydrogen, hydroxyl, straight or branched C₁-C₈ alkoxy; the N₁-oxides, the racemic mixtures, their individual enantiomers, their individual diastereoisomers, their mixtures, and pharmaceutically acceptable salts.

Preferred examples of compounds of formula (I), are those in which n is 1, R₄ is 2-aminoethyl or 3-aminopropyl, R₂ and R₃ are hydrogen (these compounds are also named herein ST1578 and ST2541, respectively).

These compounds are fully disclosed in WO 00/53607 and the skilled reaser is referred thereto.

Another preferred camptothecin derivative is a compound of formula (II)

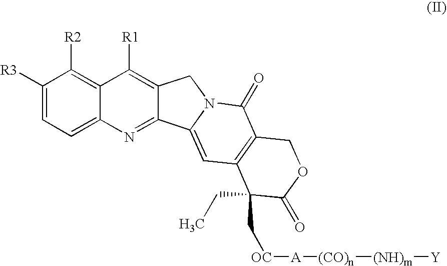

where:

A is saturated or unsaturated straight or branched C₁-C₈ alkyl, C₃-C₁₀ cycloalkyl, straight or branched C₃-C₁₀ cycloalkyl-C₁-C₈ alkyl;

when n and m are equal to 1, then Y is saturated or unsaturated straight or branched C₁-C₈ alkyl substituted with NR₁₂R₁₃ or N⁺R₁₂R₁₃R₁₄, where R₁₂, R₁₃ and R₁₄, which can be the same or different, are hydrogen or straight or branched C₁-C₄ alkyl, or Y is BCOOX, where B is a residue of an amino acid, X is H, straight or branched C₁-C₄ alkyl, benzyl or phenyl, substituted in the available positions with at least one group selected from C₁-C₄ alkoxy, halogen, nitro, amino, C₁-C₄ alkyl, or, if n and m are both 0; Y is 4-trimethylammonium-3-hydroxybutanoyl, both in the form of inner salt and in the form of a salt with an anion of a pharmaceutically acceptable acid, or Y is N⁺R₁₂R₁₃R₁₄, as defined above;

R₁ is hydrogen or a —C(R₅)═N—(O)ₚ—R₄ group, in which p is the number 0 or 1, R₄ is hydrogen or a straight or branched C₁-C₈ alkyl or C₂-C₈ alkenyl group, or a C₃-C₁₀ cycloalkyl group, or a straight or branched (C₃-C₁₀) cycloalkyl-(C₁-C8) alkyl group, or a C₆-C₁₄ aryl group, or a straight or branched (C₆-C₁₄) aryl-(C₁-C₈) alkyl group, or a heterocyclic group or a straight or branched heterocyclo-(C₁-C₈) alkyl group, said heterocyclic group containing at least one heteroatom selected from an atom of nitrogen, optionally substituted with a (C₁-C₈) alkyl group, and/or an atom of oxygen and/or of sulphur; said alkyl, alkenyl, cycloalkyl, cycloalkylalkyl, aryl, aryl-alkyl, heterocyclic or heterocyclo-alkyl groups may optionally be substituted with one or more groups selected from: halogen, hydroxy, C₁-C₈ alkyl, C₁-C₈ alkoxy, phenyl, cyano, nitro, —NR₆R₇, where R₆ and R₇, which may be the same or different, are hydrogen, straight or branched (C₁-C₈) alkyl, the —COOH group or one of its pharmaceutically acceptable esters; or the —CONR₈R₉ group, where R₈ and R₉, which may be the same or different, are hydrogen, straight or branched (C₁-C₈) alkyl; or R₄ is a (C₆-C₁₀) aryl or (C₆-C₁₀) arylsulphonyl residue, optionally substituted with one or more groups selected from: halogen, hydroxy, straight or branched C₁-C₈ alkyl, straight or branched C₁-C₈ alkoxy, phenyl, cyano, nitro, —NR₁₀R₁₁, where R₁₀ and R₁₁, which may be the same or different, are hydrogen, straight or branched C₁-C₈ alkyl; or R₄ is a polyaminoalkyl residue; or R₄ is a glycosyl residue; R₅ is hydrogen, straight or branched C₁-C8 alkyl, straight or branched C₂-C₈ alkenyl, C₃-C₁₀ cycloalkyl, straight or branched (C₃-C₁₀) cycloalkyl-(C₁-C₈) alkyl, C₆-C₁₄ aryl, straight or branched (C₆-C₁₄) aryl-(C₁-C8) alkyl; R₂ and R₃, which may be the same or different, are hydrogen, hydroxyl, straight or branched C₁-C₈ alkoxy; the N₁-oxides, the racemic mixtures, their individual enantiomers, their individual diastereoisomers, their mixtures, and pharmaceutically acceptable salts.

Preferred examples of compounds of formula (II), are those in which p is 1, R₄ is tert-butyl, the particularly preferred compound is succinyl-valyl-20-O-(7-terbutoxyiminomethylcamptothecin) (named herein ST2677).

These compounds are fully disclosed in WO 03/101996 and the skilled reaser is referred thereto.

Another preferred camptothecin derivative is a compound of formula (III) or (IV)

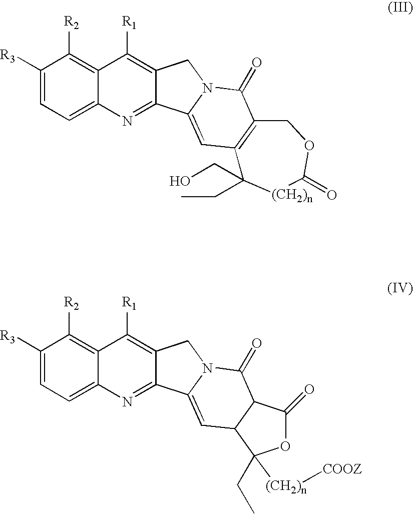

where:

R₁ is hydrogen or a —C(R₅)═N—(O)ₚ—R₄ group, in which p is the integer 0 or 1, R₄ is hydrogen or a straight or branched C₁-C₈ alkyl or C₂-C₈ alkenyl group, or a C₃-C₁₀ cycloalkyl group, or a straight or branched (C₃-C₁₀) cycloalkyl-(C₁-C₅) alkyl group, or a C₆-C₁₄ aryl group, or a straight or branched (C₆-C₁₄) aryl-(C₁-C₈) alkyl group, or a heterocyclic group or a straight or branched heterocyclo-(C₁-C₈) alkyl group, said heterocyclic group containing at least one heteroatom selected from an atom of nitrogen, optionally substituted with an (C₁-C₈) alkyl group, and/or an atom of oxygen and/or of sulphur; said alkyl, alkenyl, cycloalkyl, cycloalkylalkyl, aryl, aryl-alkyl, heterocyclic or heterocyclo-alkyl groups can optionally be substituted with one or more groups selected from the group consisting of: halogen, hydroxy, C₁-C₈ alkyl, C₁-C₉ alkoxy, phenyl, cyano, nitro, and —NR₆R₇, where R₆ and R₇, which may be the same or different, are hydrogen, straight or branched (C₁-C₈) alkyl, the —COOH group or one of its pharmaceutically acceptable esters; or the —CONR₈R₉ group, where R₈ and R₉, which may be the same or different, are hydrogen, straight or branched (C₁-C₈) alkyl; or

R₄ is a (C₆-C₁₀) aryl or (C₆-C₁₀) arylsulphonyl residue, optionally substituted with one or more groups selected from: halogen, hydroxy, straight or branched C₁-C₈ alkyl, straight or branched C₁-C₈ alkoxy, phenyl, cyano, nitro, —NR₁₀R₁₁, where R₁₀ and R₁₁, which may be the same or different, are hydrogen, straight or branched C₁-C₉ alkyl; or:

R₄ is a polyaminoalkyl residue; or

R₄ is a glycosyl residue;

R₅ is hydrogen, straight or branched C₁-C₈ alkyl, straight or branched C₂-C₈ alkenyl, C₃-C₁₀ cycloalkyl, straight or branched (C₃-C₁₀) cycloalkyl-(C₁-C₈) alkyl, C₆-C₁₄ aryl, straight or branched (C₆-C₁₄) aryl-(C₁-C₈) alkyl;

R₂ and R₃, which may be the same or different, are hydrogen, hydroxy, straight or branched C₁-C₈ alkoxy;

n=1 or 2,

Z is selected from hydrogen, straight or branched C₁-C₄ alkyl; the N₁-oxides, the racemic mixtures, their individual enantiomers, their individual diastereoisomers, their mixtures, and their pharmaceutically acceptable salts.

These compounds are fully disclosed in WO 03/101995 and the skilled reaser is referred thereto.

Another preferred camptothecin is the one disclosed in Arimondo P. B. et al., Nucleic Acid Research, 2003, Vol. 31, No. 14; 4031-4040, in particular 7-ethyl-10-hydroxycamptothecin. Still another preferred compound is 10-hydroxycamptothecin.

The ligand is selected from the group consisting of ribonucleic acids, deoxyribonucleic acids, PNAs, peptide nucleic acids, 2′O-alkyl ribonucleic acids, oligophosphoramidates, LNAs (RNAs blocked for the ribose conformation (Petersen and Wengel 2003) and is called TFO when it forms a triple helix and MGB when it binds to the minor groove. The latter are chosen from polyamides of N-methylpyrrole, N-methylimidazole and N-methyl-3-hydroxypyrrole and β-alanine.

An object of the present invention is also a compound formula I

A-B—C

wherein

A is a DNA sequence-specific ligand capable of simultaneously and specifically recognizing a sequence common to the genes of pathological interest;

B is a linker arm, said linker arm being bound to the 3′ end of A;

C is a camptothecin derivative of the above formulae (I)-(IV).

In the general teaching of the present invention, the elements A and C of the compounds above described can be connected by the linker arm through different positions of the poison molecule, provided that this position has, a suitable functional group to be bound to the ligand.

In the preferred embodiment of the present invention, using a camptothecin derivative, the ligand A can be attached to the camptothecin molecule preferably at position 7, 10 or 20.

Suitable linker arms comprise a succession of carbon and heteroatoms, selected in the group comprising N or O, of length from 1 to 50, with a preference for 2 to 30; and end terminal moieties capable of reacting to give phosphoramide or amide bonds, or thioeters.

Examples of such linker arms are diamino alkyls such as —HN'(CH₂)ₙ—NH—, wherein n is an integer from 1 to 12; —NH—(CH₂)ₙ—CO—, glycols (—O(CH₂)ₘO)ₙ—, where n is an integer from 2 to 6 and m from 2 to 3.

Examples of conjugates according to said embodiment are selected in the group comprising: TFO-L3-SCPT, and (3+3)-CPT, (4+4)-CPT, TFO-18-L6-10CPT TFO-18-L4-10CPT, TFO 16-L6-10CPT, and TFO16-L4-10CPT, TFO16-L6-7CPT, TFO18-L6-7CPT, SCPT-Ln-TFO, TFO-L4-cCPT, TFO-L6-cCPT, wherein TFO is a triple helix forming oligonucleotide, L is the number of CH₂ groups and CPT are camptothecin derivatives. (3+3) and (4+4) are hairpin polyamides.

Other conjugates comprise rebeccamycin, particularly indolocarbazole derivatives of rebeccamycin as poison.

Examples of such conjugates are TFO-Ln-RBC (Ln=—O(CH₂)₂O)ₙ—, n=2; 3 or 6.

According to another embodiment of the invention, said conjugate is a binary complex characterized in that it consists of a ligand, such as above defined, and a derivative of said topoisomerase I inhibitor, wherein the linker arm is incorporated in a substituent group of the inhibitor. Said substitution group comprises an end terminal moiety capable of reacting with a phosphate or a phosphotioate group. Examples of such conjugates are TFO-ST1578 and TFO-ST2541 and the related compounds of formulae (I)-(IV).

A third group of conjugates is characterized in that it consists of a ligand, a derivative of a said topoisomerase I poison substituted by a group playing the role of a part of the said linker, and, furthermore, a linker arm. Examples are TFO-(CH₂)ₙ-cCPT, with n an integer between 2 and 6; TFO-(CH₂)₃-SCPT, SCT-TFO, TFO-ST2677.

As above mentioned, the conjugates of the invention direct a topoisomerase I-mediated DNA cleavage in the vicinity of each oligopyrimidineoligopurine sequence of said target genes containing a number of purines between positions 2 and 30. Because of the geometry of the DNA/topo I cleavage complex, the cleavage site should be on the 3′ side of the triplex on the oligopyrimidine strand of the target.

This chemical compound is also characterized in that said cleavage site induced by the topoisomerase I inhibitor is positioned 1 to 10 nucleotides from the end of the ligand binding site.

Examples of substitution groups comprise diamino alkyl, with optionally an unsaturation, such as H₂N(CH₂)ₙ—O—N═CH— wherein n is an integer from 2 to 6 and those groups recognizable in the R₄ group of compounds of formulae (I)-(IV). Other substitution groups comprise a dicarboxylic acid chain comprising a —CO—NH-group-.

Such groups are for example

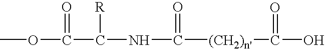

wherein R is a C1-C4 linear or branched alkyl, n′ is an integer from 1 to 6.

In conjugates of the invention, the inhibitor is for example camptothecin and the substitution group occupies position 20 thereof.

According to still another embodiment of the invention, the conjugate comprises a ligand linked to the substitution group of the inhibitor via a linker arm such as above defined.

The invention also relates to a method for the preparation of said conjugates. Phosphoramide bonds are obtained by reaction with triphenylphosphine and dipyridyldisulfide in the presence of 4-dimethylaminopyridine as described in Grimm et al. 2000; while amides bonds are formed either by this method, by carbodiimide activation of the acid function, or by modified peptide synthesis procedures upon use of HATU.

Said conjugates are advantageously effective through a new mechanism compared to cytostatic molecules. As shown by the examples, said conjugates penetrate into cells and bind their targets.

The new approach of the invention is thus aimed at maintaining this optimum antitumoral effectiveness while reducing side effects.

For example, it is established that the use of topoisomerases I inhibitors is associated, in approximately 15% of cases, with the appearance of secondary leukaemias characterized by reciprocal translocations of genes, now well characterized. Directing these inhibitors towards certain chosen genes can reduce their leukaemogenic power by allowing a better selectivity of the therapeutic effect.

The invention also relates to the use of said conjugates in a method for specifically inhibiting the expression of a gene of interest or simultaneously of several genes, this gene or these genes coding for one or more viral or pathogenic proteins, or proteins involved in the development and maintenance of the tumoral state of cells, for example.

It will be judged that, in advantageous manner, a single oligonucleotide-inhibitor conjugate can have effects analogous to the combinations of antitumoral drugs used in clinics at present. The number of sites targeted by the conjugates are highly reduced compared to CPT when used alone.

Accordingly, the invention also relates to pharmaceutical compositions characterized in that they contain an effective quantity of at least one conjugate as defined above, in combination with a pharmaceutically inert vehicle.

These compositions are advantageously in forms allowing their administration by injection or spray. The unit and daily doses will be measured by a person skilled in the art according to the type of pathology, in particular of cancer to be treated. In this connection, it will be appreciated that some of the conjugates disclosed in the state of the art were tested only in in vitro, acellular systems. The present inventors found very difficult, even not possible to administer the compounds to cells. Therefore, the compounds of the present invention, both in the aspect of new compounds and in the aspect of the use of known compounds shall be administered together with a transfection vector in acellular systems. Examples of transfection vector are nanoparticles, liposomes, cationic lipids and cationic polymers.

In a totally surprising manner, the compounds wherein C is a camptothecin derivative of formula (I)-(IV), in particular camptothecin derivatives identified with the code ST1578 and ST2677, do not need any transfection vector in order to be administered to cells, since they penetrate ex vivo cell membrane. Therefore, the compositions and drugs comprising the compounds A-B—C, wherein C is a camptothecin derivative of formula (I)-(IV), in particular camptothecin derivatives identified with the code ST1578 and ST2677, will advantageously not need a supplemental transfection vector, thus making their biological application simpler.

Other characteristics and advantages of the invention are given in the examples which follow, with reference to the scientific literature as well as to the attached drawings in which:

FIG. 1 is a diagram which illustrates the principle of targeting the cleavages/cuttings of DNA by topoisomerase I at specific sites;

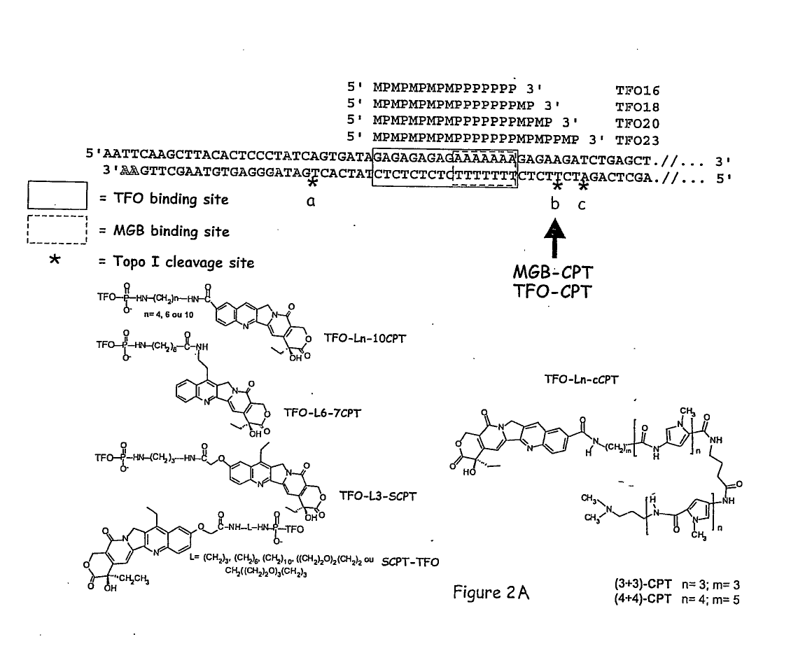

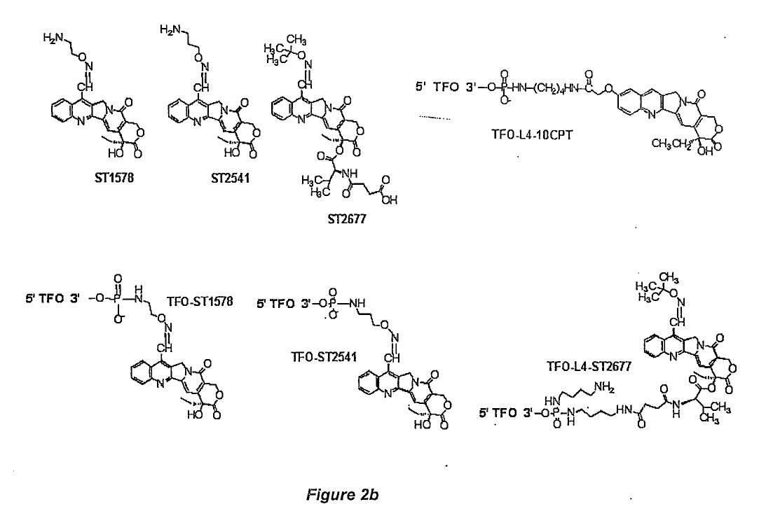

FIG. 2A illustrates a known study system: the 324-bp duplex containing a target oligopurineoligopyrimidine sequence and the sequence of the corresponding triplex-forming oligonucleotide (TFOs).

The oligonucleotides forming a triple helix (TFO16, TFO18, TFO20, TFO23), were modified in order to increase the stability of the complexes (for example, by using 5-methyl-deoxycytosines (M) and 5-propynyl-deoxyuracils (P). The TFOs were coupled to 20S-10-carboxycamptothecin (10CPT), 20S-7-aminoethylcamptothecin (7CPT), 20S-7-ethyl-10-hydroxycamptothecin acetic acid (SCPT), 20S-7-aminoethyliminomethylcamptothecin (ST1578), 20S-7-aminopropyliminomethylcamptothecin (ST2541) and to succinyl-valyl-20-O-(7-terbutoxyiminomethylcamptothecin)(ST2677).

Two minor-groove ligands, of (3+3) and (4+4) hairpin polyamide type, were coupled to 10-carboxycamptothecin (10CPT).

The binding site of the TFO16 and of 2 minor-groove ligands is indicated by squares. The oligonucleotides bind to the oligopurineoligopyrimidine sequence, forming Hoogsteen-type hydrogen bonds with the purines of the Watson-Crick base pair. The minor-groove ligands, (3+3) and (4+4), bind interacting in the minor groove. The chemical formulae of the conjugates are specified in the lower part of the figure and the linker arm is represented in italics. The oligonucleotides come from the company Eurogentec (Belgium) and are coupled with the inhibitors according to the methods described in Grimm et al. Nucleosides Nucleotides Nucleic Acids 19 (2000) pp. 1943-1965 and by adapted peptide synthesis procedures based upon use of HATU. The minor-groove ligands were synthesized as described in Arimondo et al. Angewandte Chem. Int. Ed. 40 (2001) pp. 3045-3048;

FIG. 2B represents the formulae of camptothecin derivatives ST1578, ST2541 and ST2677 and conjugates TFO-ST1578, TFO-ST2541, TFO-L4-ST2677 and TFO-L4-10CPT;

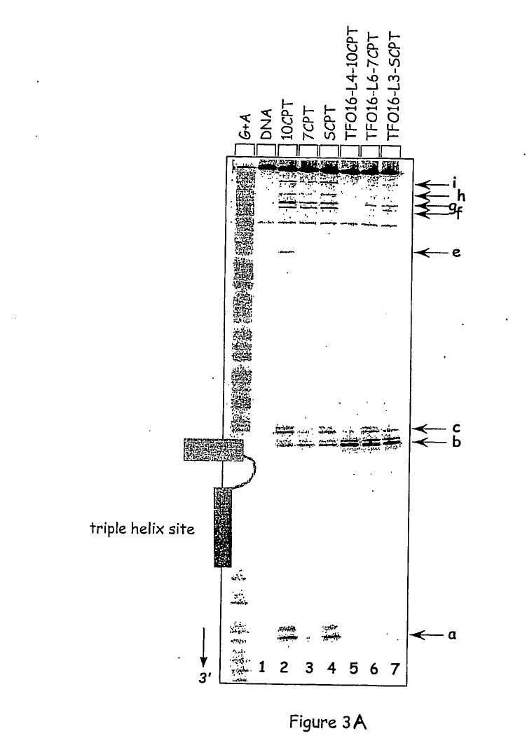

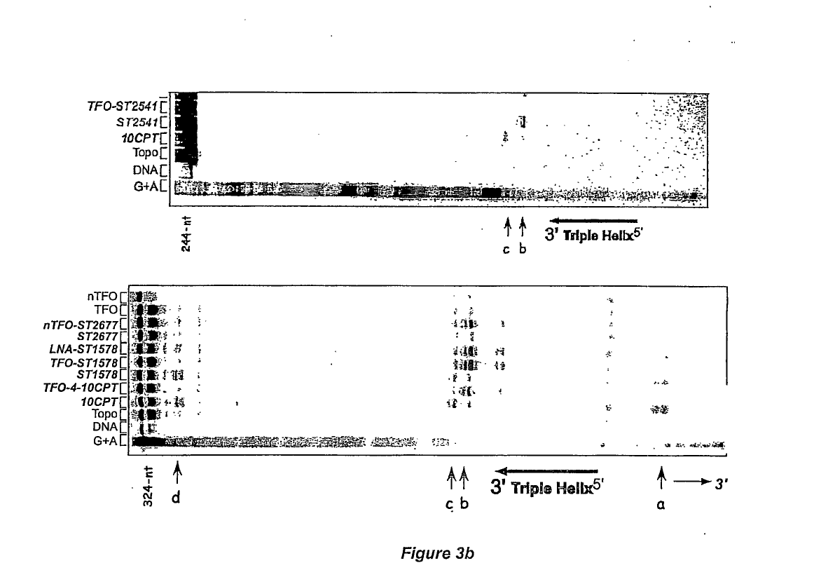

FIG. 3A represents the topoisomerase I cleavage sites. The radiolabelled 324-bp duplex in position 3′ on the oligopyrimidine strand (well 1) was incubated with topoisomerase I in the presence of three camptothecin derivatives (wells 2-4, 10CPT, 7CPT, SCPT), or of these three derivatives conjugated with TFO 16 (wells 5-7, TFO16-L4-10CPT, TFO16-L6-CPT, TFO16-L3-SCPT). The cleavage sites are indicated by letters and the binding site of the conjugate is shown diagrammatically. L3=diaminopropynyl; L4=diaminobutyl, L6=diaminohexyl; after incubation, the protein is digested by a treatment with SDS/proteinase K and the cleavage products are analysed on a denaturating gel;

FIG. 3B represents results obtained with other conjugates according to the invention wherein DNA was used as controls, in the presence of topoisomerase alone or with 5 μM of 10 CPT, of ST 1578, ST2677 or ST 2541, or 1 μM of non conjugated TFO At 1 μM, all the conjugates direct the cleavage of DNA by human topoisomerase only on the 3′ side of the binding site of the oligonucleotide, where the inhibitor is position by formation of the triple-helix (site b). nTFO bears unmodified cytidine and thymidines.

On the contrary, the inhibitor alone stimulates the cleavage at several sites.(sites a,b,c and d).

Conjugates TFO-ST1578 and TFO-ST2541 are 3 times more efficient than conjugate TFO-L4-10 CPT.

Conjugate TFO-L4-ST2677 is comparable to conjugate TFO-L4-10 CPT.

Results are also given regarding another chemically modified TFO, i.e. LNA (Locked Nucleic Acids) having sequences +CP+CP+CP+CP+CP+TP+TP+TP (wherein C designates LNA cytidine and +T=LNA thymidine).

LNA was attached in a one-step synthesis to ST1578, to give LNA-ST1678 conjugate. The effect thereof to direct the cleavage at site b was evaluated. Said conjugate was 2 times less efficient than TFO-ST1578 analog.

The molecular constraints of the DNA/topo I cleavage complex govern the geometry of the ternary complex and orient the DNA cleavage in the presence of the bound triplex-forming oligonucleotide.

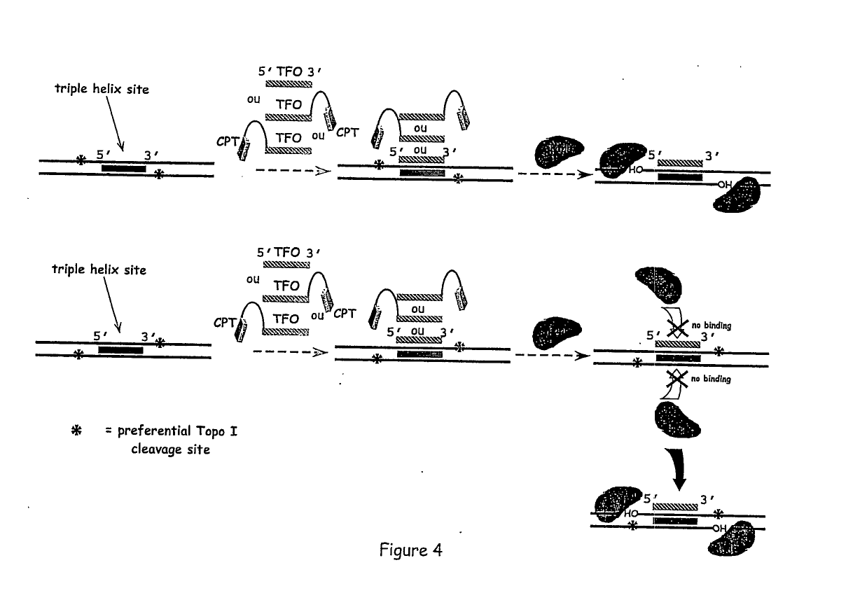

FIG. 4 shows that the presence of the triple helix induces a cleavage of the 5′ side of the triple helix on the oligopurine strand of the target and one on the 3′ side on the oligopyrimidine strand, whether this is a preferential site or not. The presence of the inhibitor on the oligonucleotide in position 3′ has the effect of amplifying the signal.

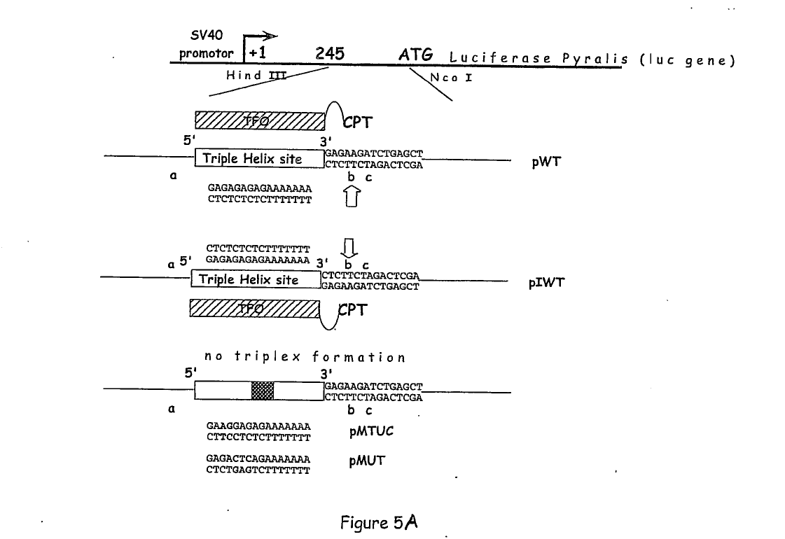

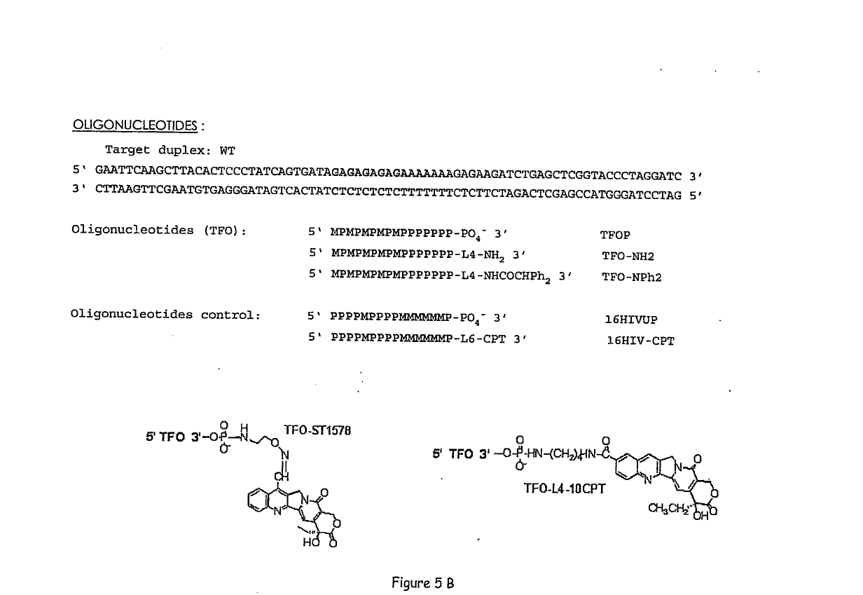

FIG. 5A represents an experimental construction: the plasmids used were obtained by cloning 54-bp duplexes at the Hind III/Nco I sites in the transcribed and non-translated region of the pGL3 Promoter vector (Promega), containing the Pyralis luciferase gene under the control of the SV40 promoter. Sequences of TFO binding and a site sensitive to camptothecine in the vicinity thereof are placed in the transcript region upward of luciferase gene of Pyralis (luc). Inserts of 54-bp comprised: intact triple-helix sequence (pWT), used in experiments in vitro; the triple-helix sequence mutated on 3 sites (pMUT); the triple-helix sequence and on the 3′ side, a well-known cleavage site stimulated by camptothecine (pTID), and the intact triple-helix sequence inserted on the opposite strands to avoid any anti-sens effect of TFO (pIWT).

FIG. 5B gives target duplexes, TFO and control oligonucleotide sequences:TFO-L4-10CPT was used as conjugate and, as control, the oligonucleotide protected in 3′ by a phosphate (compound TFOP), or by the linker arm used for the coupling of 10CPT, NH₂—(CH₂)₄—NH₂ (compound TFO-NH2). Said arm was linked to diphenylacetic acid (compound TFO-NPh2). As last controls, an oligonucleotide containing the same amended bases was used but with a different sequence, linked either to a phosphate in 3′ (16HIVUP), or to 10CPT via the linker arm NH₂—(CH₂)₆—NH₂ (compound 16HIV-CPT) Conjugate TFO-ST1578 was then compared to TFO-L4-10CPT.

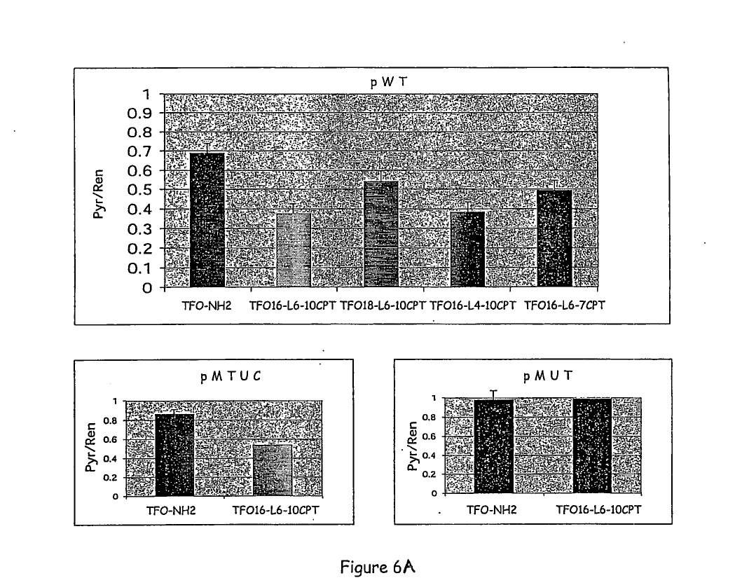

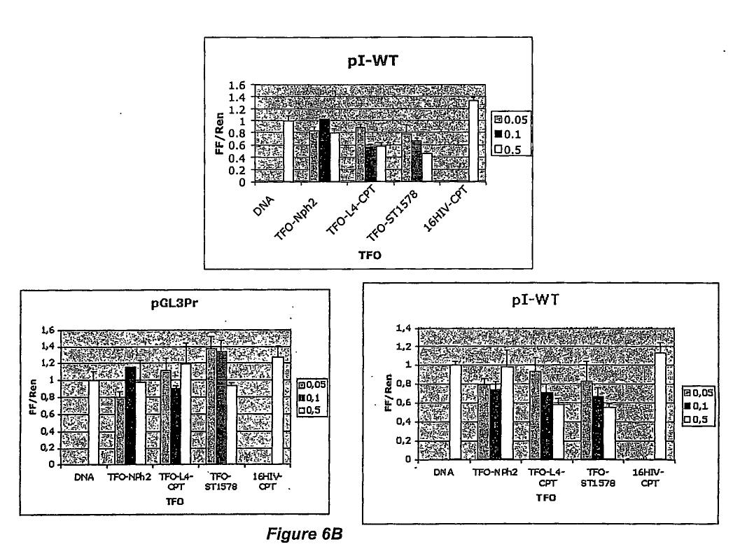

FIG. 6A illustrates for the first time with these molecules the inhibition of the transcription of the Pyralis luciferase gene in HeLa cells. Human adherent HeLa cells were cultured in DMEN (Invitrogen) supplemented with FCS 10%, at 37° C. and 10% CO₂. The cells were seeded (110000 cells/mL) in 96 wells plates at 125 μl/wells. After 24 h, the medium is changed for 112.5 μl of fresh medium and 12.5 μl of a transfection mixture. Said transfection mixture contains: 1 μg pGL3Pr or modified; 0.5 μg of pRL-TK, various concentrations of oligonucleotides and 3 μL of Superfect™ (QIAGEN) in a free serum medium. The mixtures were prepared in duplicate or triplicate. After 24 h, the cells were lysed and luciferase expression was evaluated. Dual-luciferase™ Reporter Assay System (Promega) was used to determine the activities of both reporters (Pyralis and Renilla) on the same cellular lysate: each well of 96-well plate is lysed in 30 μL of passive lysis buffer, 15 μl were analysed with “Dual-luciferase™ Reporter Assay System” with an automated apparatus (Victor/Wallac). The ratio between both activities (Pyralis and Renilla) was used to measure the selectivity of the effect. All the values of the ratio between both activities in the presence of different oligonucleotide were normalized with respect to the expression of plasmides in the absence of conjugates (DNA). The control oligonucleotides have no effect on the expression of Pyralis luciferase. Only conjugate TFO-L4-10CPT inhibits its expression from about 40-50% at 0.5 μM, on both targets which contain the intact triple-helix sequences (pTID and pWT).

On the commercial plasmide which has no insert, pGL3Pr, the conjugate has no effect and the effect is highly reduced on the one which has a mutated triple-helix (pMUT);

FIG. 6B relates to results obtained when using a plasmid construction with reversed strands.

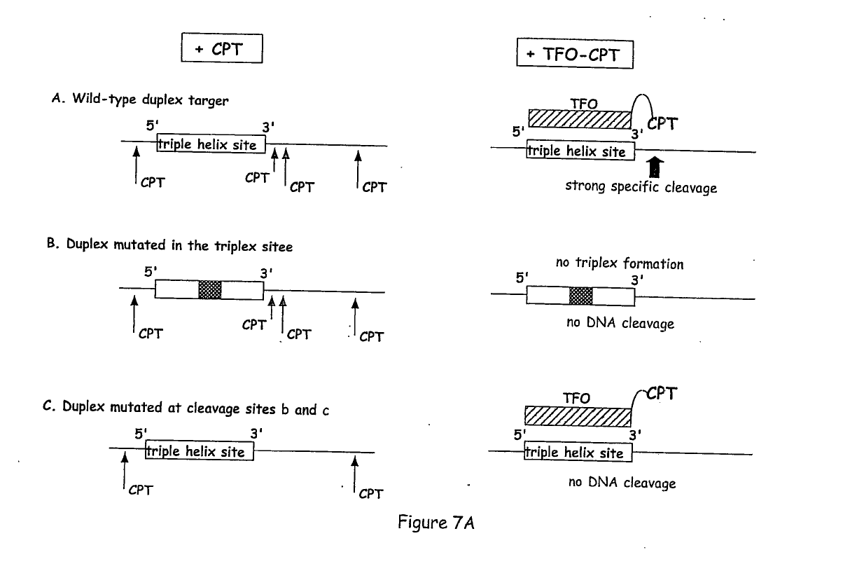

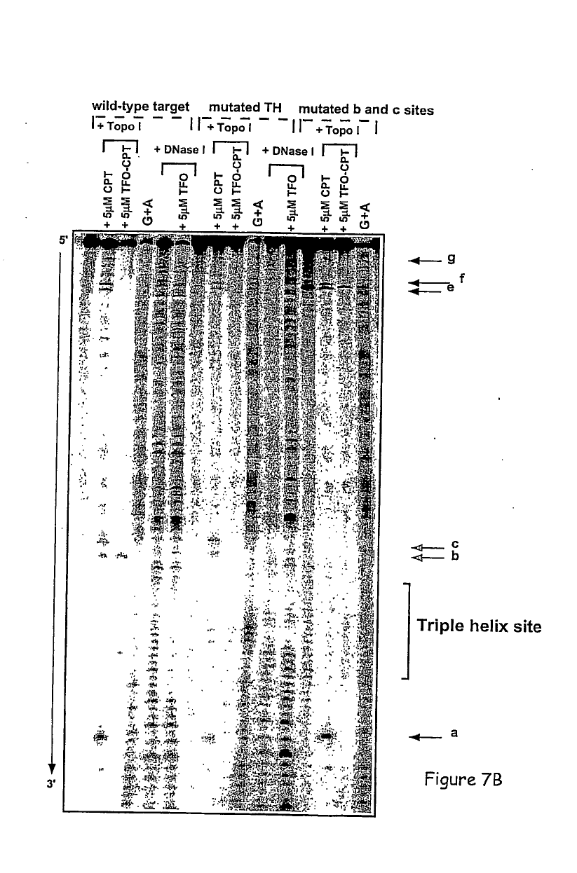

FIGS. 7A and 7B show the formation of a triplex and the presence of a strong specific break in the presence of the conjugate (Example A) and a contrario the absence of formation of the triplex and of a specific cleavage of the DNA in the case of mutation on the triple helix site (Example B) and the formation of a triplex but the absence of a strong topo I-mediated DNA cleavage sites at the 3′ end of the triplex site in the case of a mutated duplex at the cleavage sites b and c (Example C).

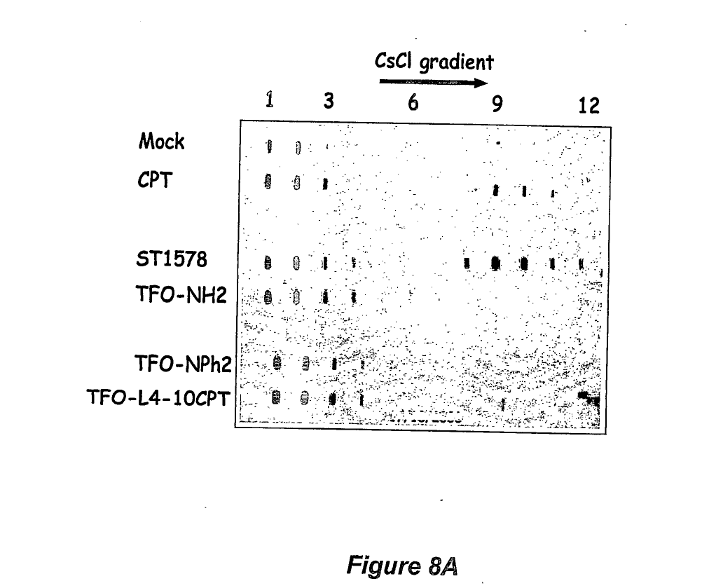

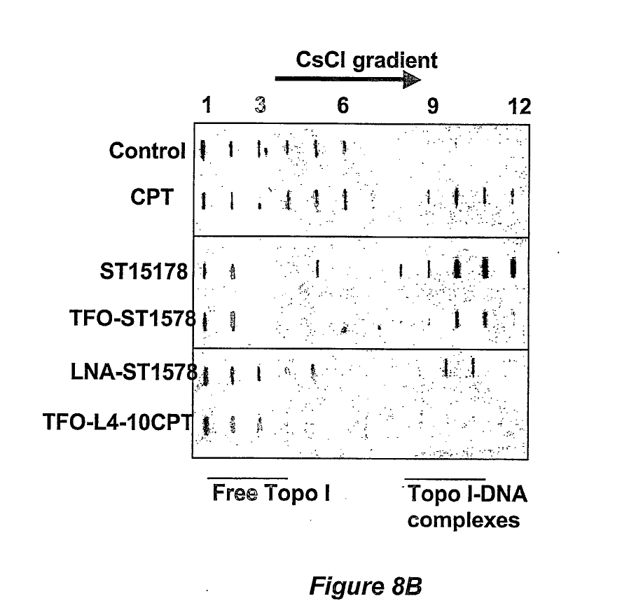

FIG. 8 gives correlation results of the biological effects with the formation of DNA/topo I/CPT complexes: the formation of the complexes in the cells was followed by immunoblot.

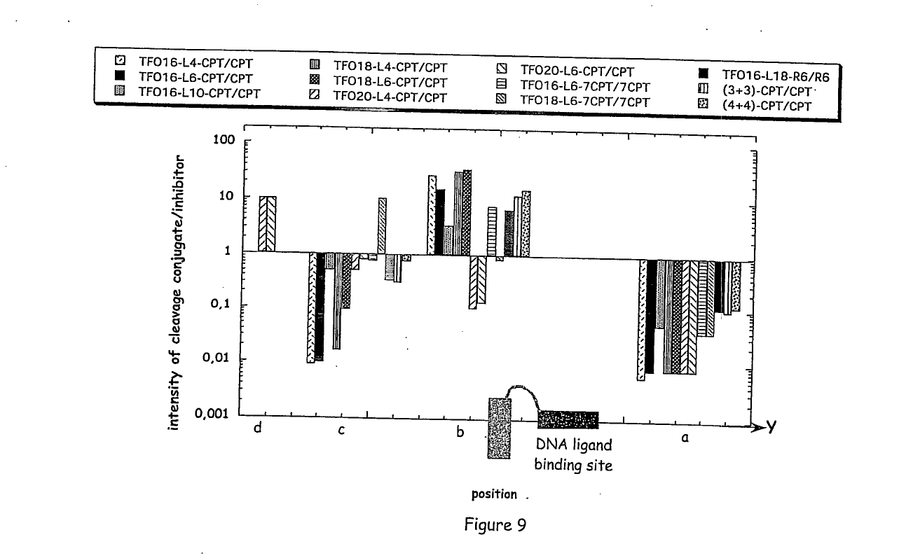

FIG. 9 illustrates the effectiveness (in terms of cleavage intensity compared with the inhibitor alone and of a given site a, b, c and d) of certain conjugates/complexes which are useful in the method of the invention.

By conjugating a topoisomerase inhibitor to an oligonucleotide capable of specifically recognizing a DNA sequence, it is possible to target the inhibitor on a group of chosen genes, thanks to the formation of a specific triple helix complex on a target sequence common to the genes chosen. It then becomes possible to selectively induce the irreversible lesions on these genes and to inhibit their expression.

This can be achieved in a manner known per se, in particular, using the covalent coupling of topoisomerase I inhibitors with sequence-specific DNA ligands, such as oligonucleotides, or non-nucleic ligands such as minor-groove ligands (polyamides composed of N-methyl pyrroles and imidazoles) or also zinc finger peptides.

In fact, such ligands can specifically recognize certain DNA sequences by binding, respectively, in the major and minor grooves of the double helix. The chemical coupling of topoisomerase I inhibitors to these DNA ligands selectively positions the inhibitor in the vicinity of the binding site of the ligand and thus specifically directs to this site the breaks induced by topoisomerase I.

The inventors thus developed a new concept based on the targeting of topoisomerase inhibitors to a gene or group of genes selected for their involvement in the proliferation and maintenance of the tumoral state of cells. These genes are chosen, for example, from genes controlling the cell cycle and division, proliferation, and from anti-apoptotic genes. Viral genes can also be targeted with this strategy.

Depending on the length of the oligonucleotide chosen, the selectivity can be modulated, in order to be aimed at only a single gene, or loosened, in order then to inhibit a group of genes.

This innovative strategy in antitumoral chemotherapy can be extended to other pathologies where the simultaneous inhibition of several genes/functions would be of evident therapeutic interest.

The usefulness of the pharmacochemical approach which will be described below resides essentially in the definition of a new “bicephalous” methodology with a conjugate having 2 heads, one recognizing the DNA of the target, the other recruiting the topoisomerase.

The design of these compounds must be adapted to the sequence aimed at and must have the characteristics described above.

The pharmacogenic approach involves the development of a new therapeutic strategy based on the targeting of topoisomerase I poisons towards specific genes, involved in the cell proliferation and maintenance of cancerous tumours.

Said approach consists of chemically coupling topoisomerase I inhibitors to modified or non-modified oligonucleotides, capable of binding selectively by formation of stable triple helices on genes involved in particular in cell growth and/or on anti-apoptotic genes, angiogenesis (FIG. 2: targeting of topoisomerase I-mediated DNA cleavage by an oligonucleotide-inhibitor conjugate).

The DNA ligand approach offers the possibility of acting simultaneously on the expression of several genes, choosing a target sequence common to these genes.

In order to effectively treat a multigenic pathology such as cancer, it is in fact essential to simultaneously control gene families, and more precisely, a group of genes which alter the normal proliferative circuits of cells.

Thus, in highly advantageous manner, a single oligonucleotidic-inhibitor conjugate could have effects analogous to the combinations of anti-tumour drugs currently used in clinics.

This approach can make it possible to maintain this optimum antitumoral effectiveness while reducing certain side effects. For example, it is an established fact that the use of topoisomerase II inhibitors is associated, in approximately 15% of cases, with the appearance of secondary leukaemias characterized by reciprocal gene translocations, now well characterized. Directing these inhibitors towards certain chosen genes can reduce their leukaemogenic power by allowing a better selectivity of the therapeutic effect.

Also in one of its particularly essential aspects, the present invention also relates to a method which makes it possible to direct the action of topoisomerase I inhibitors towards a DNA-specific site making it possible to induce, selectively at this site, cleavage by topoisomerase I.

This new concept is detailed hereafter purely by way of illustration and non-limitatively, taking two groups of genes involved in the development and maintenance of cancer (1) the genes of a survival route, such as that which is established when the growth factor IGF-1 (insulin-like growth factor-1) binds to its receptor (IGF-1R) and (2) the genes which inhibit apoptosis, such as IAPs and the anti-apoptotic genes of the Bcl-2 family. These genes are overexpressed in certain cancers and blocking them leads to an antitumoral effect.

The inventors carried out a search for sequences capable of forming triple helices and common to the group of genes of interest to be targeted. This search was carried out using the GCG software Unix findpatterns program (Genetics Computer Group, Infobiogen, Villejuif).

In a preliminary search, the inventors identified an oligopyrimidine sequence, comprising 12 base pairs (bps), that is common to the IGF-1, IGF-1R and AKT/PBK genes and a 10-bp sequence common to the bcl-2, bcl-X_{L} and survivine anti-apoptotic genes.

Moreover the TFO sequence described in FIG. 2 binds to the list of genes reported in Table 1. While free CPT derivatives induce cleavage with little specificity in the genome (and thus at many sites), the TFO-poison conjugate with the base sequence depicted in FIG. 2 induce cleavage only on these genes, and, among them, in particular, IGF1R and VEGF, involved in tumor proliferation and maintenance. The search was made with the use of publicly available bioinformatics resources at UCSC.

As already mentioned, non-nucleic ligands of sequence-specific DNA, such as the minor-groove ligands (polyamides composed of N-methyl pyrrole and N-methyl imidazoles) can also be used in order to direct the action of topoisomerase inhibitors towards a given site.

Their use should make it possible to be free of the oligopyrimidineoligopurine target sequence restriction imposed by the formation of a stable triple helix.

Results with minor-groove ligands coupled with camptothecin are presented hereafter.

This search for a sequence common to a group of target genes should make it possible to define the optimum target sequence, chosen in such a manner as to form part exclusively, or chiefly, of the group of selected genes.

In cases of the use of triple helix oligonucleotides, the cleavage by the conjugates is directed onto each oligopyrimidine oligopurine target sequence containing a number of purines from 2 to 100, preferably 10-30, with a cleavage site induced by the topoisomerase I inhibitor on the 3′ side of the triplex on the oligopyrimidine strand of the target.

Moreover, the cleavage site induced by the inhibitor and advantageously positioned 1 to 10 nucleotides from the triple helix end and the linker arm is adapted according to the cleavage site, the inhibitor used and the point of attachment of the inhibitor to the oligonucleotide.

As regards the oligonucleotide-topoisomerase inhibitor conjugates, the inventors carried out the coupling to camptothecin derivatives, topoisomerase inhibitors. In a preliminary work, the inventors showed that the covalent coupling of camptothecin and rebeccamycin derivatives, which are topoisomerase I inhibitors, to an oligonucleotide 16 nucleotides long, directs in vitro the cleavage by topoisomerase I specifically to the site where the inhibitor is positioned by formation of the triple helix (Arimondo et al., 1999, 2000a).

The same step can be carried out with other types of inhibitors, which are topoisomerase poisons which can be attached, in the same manner, namely in covalent fashion to the end of DNA-specific ligands.

The optimization of the linker arm which unites the ligand part and the inhibitor part is very important and must be adapted according to the position of the cleavage site of the inhibitor used in respect with the ligand binding site and the point of attachment of the inhibitor to the oligonucleotide (Arimondo et al. 2002).

After the synthesis of the oligonucleotide-inhibitor conjugates, and before the evaluation of their cell activity, their ability to form a triple helix—by gel shift experiments and thermal dissociation experiments—should be analyzed. For example, TFO of composition described in FIG. 2 binds and directs topo I-mediated DNA cleavage in vitro specifically to the ligand recognition site in two genes tested, sharing the same target sequence.

Cell Activity of the Inhibitors Selected

With regard to the activity of the oligonucleotide-inhibitor of topoisomerase I conjugates, molecular and cell systems make it possible to study the effect of the different conjugates on the cascade of the genes involving IGF-1 and its receptor (Hamel et al., 1999). In particular, the cleavage activity can be evaluated by direct analysis of the genomic DNA, and the action specificity by transcriptome (DNA chips and Northern blot) and proteome (bi-dimensional gel and Western blot) analyses. As the IGF-1 and IGF-1R genes are involved in the proliferation of glioblastomas, hepatocarcinomas and tumours of the prostate, their inhibition by antisense constructions blocks the proliferation of tumours grafted onto animals (Lafarge-Frayssinet et al., 1977). Tests on tumorous cells in culture will make it possible to select the most effective oligonucleotide conjugates, and to use an animal model (for example with glioblastomas injected into nude mice or hepatocarcinomas in syngenic rats).

The pharmacokinetics of the conjugates can also be evaluated with standard procedures.

As regards the most effective conjugates, their ability to inhibit the proliferation of cancerous cells can for its part be evaluated by using different tumoral cell lines then, for the most cytotoxic molecules in vitro, on in vivo models, from human tumours xenografted into mice.

### Examples of Industrial Applications of Certain Aspects and Aims of the Present Invention

Evaluation of DNA ligands coupled with topoisomerase I inhibitors as anticancer agents.

The economic stakes are considerable since new therapeutic routes in pathologies as important as cancers are involved.

Thus the identification of deregulated genes in pathologies can form the basis of new pharmacogenomic products.

It is evident that pharmacogenic successes will have major consequences for:
the reduction of side effects and the increase of the treatment efficiency
the reduction in costs associated with pharmaceutical development
the development of a greater number of therapeutic solutions suited to patients
a notable reduction in public health expenditure.

This economic impact will be very substantial in the field of cancers where there is a choice between numerous therapeutic protocols with individual effectiveness levels that are unfortunately low.

### EMBODIMENTS

Abbreviations

CPT=camptothecin; P=5-propynyl-2′-deoxyuridine; M=5-methyl-2′-deoxycytidine; R=oligopurine strand of the duplex, Y=oligopyrimidine strand of the duplex.

=pairing of Watson-Crick bases

Topo=topoisomerase

Material and Methods

Inhibitors

All the inhibitors are dissolved in dimethylsulphoxide and then diluted in water. The final concentration of dimethylsulphoxide never exceeds 0.3% (v/v) in all the tests. The inhibitors are bound to the 3′ or 5′ end of the TFO as already described in FIG. 2.

The camptothecin derivatives are synthesized according to the techniques described in Arimondo et al. (2002) and in Villemin et al. (1996).

Oligonucleotides and DNA Fragments

The oligonucleotides are marketed by Eurogentec and purified on “quick spin” columns and Sephadex G-25 fine (Boehringer, Mannheim). The concentrations are measured spectrophotometrically at 25° C. using molar extinction coefficients at 260 nm calculated from the closest model (Cantor et al., 1970).

Synthesis of CTP Conjugates

Derivatives of camptothecin CPT are conjugated to the through different linker arms to the phosphate at the 3′ or 5′ end of the oligonucleotide or to the minor-groove ligand, N,N-dimethyl-N′{1-methyl-4-[1-methyl-4-[1-methyl-4-[4-{[1-methyl-4-[1-methyl-4[1-methyl-4-(4-aminobutiryl)aminopyrrol-2-carbonyl]aminopyrrol-2-carbonyl]aminopyrrol-2-carbonyl]}aminobutiryl]aminopyrrol-2carbonyl]aminopyrrol-2-carbonyl]aminopyrrol-2-carbonyl}propylendiamine (3+3), according to the techniques described in Grimm et al. Nucleosides, Nucleotides (2000) with slight modifications and, for amide bonds formation, to peptide synthesis procedure using HATU adapted to oligonucleotides. The linker arms are bound by reaction of the amino-terminal end to the phosphorylated oligonucleotides at the 3′ or 5′ ends activated by treatment with N-methylimidazole, dipyridyl disulphide and triphenylphosphine as described in Arimondo et al. (2001) Angewandte Chem. above Arimondo (2002). The conjugates are characterized by UV spectroscopy and mass spectroscopy.

When no linker arm is used as in ST1578 and ST2541, the amino group on the CPT derivative is directly attached to the terminal phosphate of the oligonucleotide according to the technics described in Grimm et al. 2000.

Preparation of the (DNA) Target Genes

The pBSK(+/−) plasmid is marketed by Promega (USA) and the 77-bp target duplex is inserted between the BamHI and EcoRI sites. The digestion of the plasmid by PvuII and EcoRI produces a 324-mer fragment suitable for a labelling at the 3′ end by the Klenow polymerase (Ozyme, GB) and α[32P]dATP (Amersham, U.S.A.). Details of the techniques for the isolation, purification and labelling of this duplex DNA are described in (Arimondo 2002). The two 59-bp duplexes are obtained by labelling of a strand by a terminal transferase (Ozyme, GB) and α[32P]ddATP (Amersham, U.S.A.), followed by a hybridization with the non-labelled complementary strand for 5 minutes at 90° C. and by slow cooling to ambient temperature. The radiolabelled fragments are purified by gel chromatography as previously described (Arimondo 2002). The nomenclature of the strands is as follows: R strands for oligopurine and Y for oligopyrimidine strands.

Topoisomerase Cleavage Tests

The radiolabelled duplexes (50 nM) are incubated for 1 hour at 30° C., in 50 mM. Tris-HCl, pH-7.5, 60 mM KCl, 10 mM MgCl₂, 0.5 mM DTT, 0.1 mM EDTA and 30 μg/μL BSA, in the presence of the TFO or MGB, at the concentration mentioned (total reaction volume 10 μl). In order to analyze the Topo I DNA cleavage products, 10 units of the enzyme (Invitrogen Inc) are added, pre-incubated as described above either with the ligand and/or the inhibitors, followed by an incubation for 20 minutes at 30° C. The Topo I-DNA complexes are dissociated by addition of SDS (final concentration 0.25%). After ethanol precipitation, all the samples are re-suspended in 6 μl of formamide, heated to 90° C. for 4 minutes and cooled again on ice for 4 mins, before being deposited on 8% and 10% denaturing polyacrylamide gel [19/1 acrylamide:bisacrylamide], for the long and short targets respectively, containing 7.5 M urea in 1× TBE buffer (50 mM Tris-HCl, 55 mM boric acid, 1 Mm EDTA), In order to quantify the cleavage intensity, the gels are scanned with a Dynamics 445SI Phosphorimager. In order to determine the cleavage rates, a standardization with respect to the total deposition is carried out.

The chemical formulae of 20S-(7-ethyl-10-hydroxycamptothecin) acetic acid (SCPT), a new CPT derivative used in the preparation of TFO-SCPT and SCPT-TFO conjugates bound to the acid at position 10, as well as those of other conjugates attached either at position 10 or at position 7 such as TFO-10CPT, and TFO-7CPT, (3+3)-CPT and (4+4)-CPT for example are given in FIG. 2A. Camptothecin derivatives ST1578 and ST2677 having a substitution group playing the role of a linker arm are given in FIG. 2B.

The inventors validated the approach by chemically coupling three rebeccamycin derivatives, similar to molecules currently undergoing clinical trials as antitumoral agents, and six camptothecin derivatives with the TFOs (triple helix-forming oligonucleotides), and the 10-carboxycamptothecin derivative with two minor-groove ligands (MGB, minor groove binder) (FIG. 2).

The inventors covalently bound the inhibitors to one end of the oligonucleotides or minor-groove ligands via appropriate linker arms, when not present on the inhibitor derivative or when not long enough. The conjugates were characterized by UV spectroscopy and mass spectrometry (Q-star I). The cleavage specificity of the conjugates was measured in vitro by a standard topoisomerase I cleavage test. The cleavage index is calculated as the relationship between the cleavage intensity in the presence of the inhibitor coupled with the DNA ligand and that in the presence of the non-bound inhibitor. An example of targeting is shown in FIG. 3. The three non-coupled camptothecin derivatives (wells 2,3,4) stimulate cleavage at several sites (sites a-i). When the derivatives are covalently bound to the 3′ end of the TFO with an appropriate arm, the triple helix is formed (wells 5,6,7), and the conjugates induce cleavage only on the 3′ side of the triple helix (site “b”). This is due to the specific positioning of the inhibitor on the 3′ side of the triple helix site by binding of the oligonucleotide part of the conjugate to its target. The presence of the ligand, negatively charged in the case of the oligonucleotides, prevents the binding of the conjugated inhibitor to the other sites, as shown clearly by the disappearance of site “a” which is situated on the 5′ side of the triple helix or of other sites situated at a greater distance from this site.

The inventors demonstrated this targeting of topoisomerase [symbol] mediated DNA cleavage by topoisomerase I in the vicinity of the binding site of the DNA ligand for TFOs of different lengths (16 18, 20 and 23 nucleotides) (see FIG. 9), and for different rebeccamycin and camptothecin derivatives. The same approach was extended to other sequence-specific DNA ligands, such as N-methyl pyrrole hairpin polyamides, which bind specifically in the min [symbol] groove of DNA (FIG. 2: (3+3)-CPT and (4+4)-CPT conjugates). T [symbol] inventors also extended it to another target: the PPT (polypuri [symbol] tract) of the HIV-1 virus (5′ AAAAGAAAAGGGGGGA 3/ [symbol] TTTTCTTTTCCCCCCT 5′) and to a 22-mer sequence present in the promotor 1 of IGF-1 (5′ GAAGAGGGAGAGAGAGAGAAGG 3′/TCTTCTCCCTCTCTCTCTCTTCC 5′). Furthermore the TFO described [symbol] FIG. 2 was demonstrated to bind to intron 2 of IGF1R, (Table 1).

The approach is therefore valid in particular for two classes of sequence-specific DNA ligands (TFO and MGB), for different classes of topoisomerase I inhibitors and also for different targets.

A subject of the present invention is also a method as defined above in which, in advantageous manner the ligands used are chosen from the group constituted by sequence-specific DNA ligands, such as oligonucleotides, or non-nucleic ligands, such as minor-groove ligands (hairpin polyamides composed of N-methyl pyrroles and N-methyl imidazoles, in particular (3+3)-CPT and (4+4)-CPT conjugates) or also zinc finger peptides.

The inventors also demonstrated that the topoisomerase I cleavage efficiency thus stimulated at the binding site of the ligand depends, on one hand on the size of the linker arm between the inhibitor and the ligand and, on the other hand, on the intrinsic effectiveness of the inhibitor. Moreover the inventors observed that positioning of the antitumoral agent by binding of the ligand has the effect of increasing in vitro the local concentration of this molecule at the targeted site; in fact, the conjugates stimulate cleavage by topoisomerase I at concentrations of 1-10 nM. Moreover, the DNA/topoisomerase/inhibitor cleavage complex is much more stable when the inhibitor is conjugated to a TFO and the triple helix is formed. High concentrations of salts (>600 mM NaCl) are necessary in order to dissociate it.

This approach, where the action of these antitumoral agents is directed selectively towards the sites, the sequence of which is recognized by binding of the DNA ligand of the ligand-inhibitor conjugate, allows a radically new approach in the development of new antitumoral drugs.

Given that at present the structure of the ternary topoisomerase I/DNA/inhibitor complex has not yet been entirely explained, the inventors used the conjugates for the structural analysis of the ternary DNA/topoisomerase/inhibitor complex. Changing the point of attachment of the inhibitor to the TFO modifies the orientation of the inhibitor in the ternary complex and thus the effectiveness of cleavage by the enzyme (see FIG. 4 and 9). The inventors therefore covalently bound two camptothecin derivatives, 10-carboxycamptothecin and 7-aminoethylcamptothecin, to TFOs of different lengths. The study of the position and cleavage intensity in the vicinity of the ternary complex thus demonstrated that the current models which describe the ternary complex are not suitable and that other conformations must be taken into account. Another indication of the conformational flexibility of the ternary complex comes from the fact that the cleavage effectiveness is comparable whether the 10-carboxycamptothecin is linked to a major groove ligand (the TFO) or to a minor-groove ligand (the MGB).

Unexpectedly, the presence of the triple helix itself, alone, induces a certain targeting of topo I-mediated DNA cleavage.

The inventors demonstrated that cleavage takes place when the conjugates have the characteristics described below:

Also a subject of the present invention is first of all a method for simultaneously inhibiting the expression of several target genes coding for proteins, in particular involved in the development and maintenance of tumors, comprising the steps of:
(iv) directing the action of at least one topoisomerase I inhibitor towards a site specific to said genes by conjugating said at least one topoisomerase inhibitor to at least one DNA sequence-specific ligand capable of simultaneously and specifically recognizing a sequence common to said target genes,
(v) recognition by the said ligand of the said conjugate of the said genes in the genome and obtaining the binding of said ligand to said targets,
(vi) induction of topoisomerase I-mediated DNA cleavage, and inhibiting the expression of the said genes.

The stage of bringing together is carried out in vitro with a biological sample containing said genes and a topoisomerase, ex vivo with cells from a culture.

The presence of a topoisomerase inhibitor amplifies in advantageous manner the effect of targeting of DNA cleavage mediated by topoisomerase I. This cleavage induced by the triplex is dependent on a precise geometry: the binding of the oligonucleotide to its target stimulates cleavage only on the 3′ side of the triple helix on the oligopyrimidine strand of the target and on the 5′ side on the oligopurine strand of the target (FIG. 4).

The present invention also relates to a complex of at least one ligand, in particular a complex of a triple helix formed with an oligonucleotide (“TFO”) which induces cleavage by topoisomerase I on the 5′ side on the oligopurine strand of the target and on the 3′ side on the oligopyrimidine strand of a target gene.

The present invention moreover relates to a pyrimidine oligonucleotide forming a triple helix and coupled in position 3′ to a topoisomerase I inhibitor which stimulates a selective and strong cleavage of the enzyme on the 3′ side of the triple helix.

The 3′ side of the triplex is defined as the 3′ side of the oligopurine sequence recognized by the TFO by formation of hydrogen bonds. This orientation of the cleavage is linked to the fact that the binding of the topoisomerase I on the DNA at the cleavage site is not symmetrical and that the enzyme forms a phosphorotyrosyl bond with the 3′ phosphate of the cleaved strand leaving a 5′OH end. The triple helix can therefore be present on the 3′ side of the cleavage site on the target without steric hindrance for the enzyme. It must be stressed that not only preferential sites of topoisomerase I are induced by the presence of the triple helix, but also sites detectable only in the presence of the triple helix. It can be imagined that this is due to the local change of conformation of the DNA linked to the presence of the triplex. It must in fact be noted that cleavage effectiveness is not identical on the 5′ side and on the 3′ side of the triple helix and that it is known that the two ends of the triple helix are not equivalent. On the other hand, it could also be imagined that the enzyme's advance is stopped by physical blocking by the triplex structure which causes the enzyme to “pause” and gives it time to cleave in this vicinity. The two hypotheses are not mutually exclusive.

A subject of the present objection is also a method as defined above comprising the steps of:
(vii) directing the action of at least one topoisomerase I inhibitor towards a site specific to said genes by conjugating said at least one topoisomerase inhibitor to at least one DNA sequence-specific ligand capable of simultaneously and specifically recognizing a sequence common to said target genes,
(viii) recognition by the said ligand of the said conjugate of the said genes in the genome and obtaining the binding of said ligand to said targets,
(ix) induction of topoisomerase I-mediated DNA cleavage, and inhibiting the expression of the said genes.

According to a preferred embodiment of the method of the invention, the targeted sequence contains the site recognized by the ligand, which, in the case of the oligonucleotides, is each oligopyrimidineoligopurine target sequence containing a number of purines of 2 to 100, preferably 2 to 30 base pairs.

In still more preferred manner, said targeted sequence also comprises the site of the topoisomerase inhibitor in its vicinity in order to obtain greater effectiveness. The cleavage site induced by the inhibitor must be positioned from 1 to 10 nucleotides from the end of the triple helix. The linker arm must be adapted according to the cleavage site, the inhibitor used and the point of attachment of the inhibitor to the oligonucleotides.

The inventors then showed for the first time the validity of the approach in cells.

As in vitro experiments cannot take account of the nuclear barrier, the structure of chromatin and the specificity of the conjugates in the nucleus, the inventors tested the conjugates in cell systems. The conjugates induce a specific effect in the cells which depends on the formation of the triple helix and on the presence of the inhibitor coupled to the oligonucleotide.

More precisely, the inventors used plasmid expression vectors, transfected into the HeLa cells, where the binding sequence of the TFO and that of a site sensitive to camptothecin in its proximity are placed in the transcribed region upstream of the Pyralis luciferase gene (luc). The plasmids were obtained after cloning of fragments with 54 base pairs, containing the sequences described in FIG. 5, in the vector pGL3 Promoter (Promega) between the Hind III and Nco I sites. pRL-TK (Promega), coding for the Renilla luciferase gene, is used as transfection control.

The HeLa human adherent cells are cultivated in DMEM medium (Invitrogen) supplemented with 10% FCS, at 37° C. and 10% CO₂. The cells are seeded (110,000 cells per mL) on 96-well plates at 125 μl per well. 24 h later, the medium of the cells is replaced by 112.5 μl of fresh medium with serum and 12.5 μl of transfection mixture. The transfection mixture contains: 1 μg of pWT or pMTUC or pMUT or pIWT; 0.5 μg of pRL-TK, variable concentrations of oligonucleotides, and 3 μl of Superfect™ (Qiagen) in medium without serum. The mixtures are prepared in duplicate or triplicate. 24 h later, the cells are lysed for luciferase expression assay.

The “dual-Luciferase™ Reporter Assay System” (Promega) was used for the determination of the activities of the two reporters (Pyralis and Renilla) on the same cell lysate: each well of a 96-well plate is lysed in 30 μl of “passive lysis buffer”, 15 μl are analyzed with the “dual-Luciferase™ Reporter Assay System” kit using an automated apparatus (Victor/Wallac).

The ratio of the two activities (Pyralis/Renilla) is used to measure the selectivity of the effect. FIG. 6 shows the ratios between the two activities in the presence of different oligonucleotides, standardized compared with the expression of the plasmids in the absence of conjugates. The three plasmids pWT, pMTUC and pMUT are represented as well as 4 conjugates which differ in the length of the oligonucleotide part, the length of the arm and the bound camptothecin derivative. The oligonucleotide TFO16 bound in position 3′ to a (CH₂)₄—NH₂ (oligo-NH₂) arm is used as a control. This oligonucleotide forms a very stable triple helix.

The presence at 1 μM of the control oligonucleotide oligo-NH2, which forms a triple helix, inhibits the expression of luciferase gene by approximately 30%. Coupling to the camptothecin increases the inhibition effect (between 45% and 60% inhibition according to the conjugates). This increase in inhibition can be explained by a cleavage of the DNA in the vicinity of the triple helix site induced by the topoisomerase in the presence of camptothecin positioned by formation of the triple helix, as observed in vitro. The conjugates differ in their effectiveness: the derivatives of the 10-carboxycamptothecin TFO16, TFO16-L6-10CPT and TFO16-L4-10CPT, are the most effective (approximately 60% inhibition) (See FIG. 9). The length of the binding arm does not greatly influence the effectiveness of inhibition. In vitro experiments show that these conjugates effectively stimulate cleavage at site “b” 4 bps from the 3′ end of the triple helix (see above, FIG. 3). The TFO18-L6-10CPT conjugate, equally effective in vitro but less specific than the 16-mers, inhibits only 45% of the luciferase gene expression. The TFO16-L6-7CPT conjugate, containing 7-aminoethylcamptothecin, is less effective than the corresponding TFO16-L6-10CPT conjugate, with approximately 50% inhibition. This is in agreement with the in vitro results for cleavage effectiveness of the inhibitors: 10-carboxycamptothecin stimulates cleavage of the DNA by topoisomerase 1 more effectively than the 7-aminoethyl-camptothecin. The effect observed is surely due to the formation of the triple helix on the target by the oligonucleotide part of the conjugate. This is confirmed by measurements on the mutated targets in the triple helix sequence on two (pMTUC) or three (pMUT) sites. The presence of two purine mutations reduces the effectiveness of the inhibition, the triple helix is still formed, but less effectively: the oligo-NH2 passes from 30% inhibition to approximately 15%, and the TFO16-L6-10CPT conjugate from 60% to 45%. The presence of three pyrimidine mutations in the binding site means a total loss of inhibition. See FIGS. 7A and 7B.

To avoid an antisens effect of the conjugates on the synthetized RNA (pIWT), a plasmid construction with reversed strands was used. The results are given on FIG. 6B. The controls did not inhibit the expression of luciferase Pyralis and conjugate TFO-L4-CPT inhibits at 40-50% its expression at 0.5 μM. Conjugate TFO-ST1578 is still more efficient and an inhibition of 50-60% at 0.5 μM is measured. Said conjugates were inactive on the plasmid pGL3Pr construction which does not have the triple helix site.

In the experiments corresponding to FIG. 8, HeLa nucleus cells (5000000) were prepared and incubated 3 h at 37° C. with the topoisomerase I poison free (CPT or ST1578) or coupled to oligonucleotide (TFO-L4-10CPT or TFO-ST1578 or LNA-ST1578), or with a control oligonucleotide (TFO-NH2 or TFO-NPh2) at various concentration (in FIG. 8 at 5 μM). After adding of sarkosyl, the lysates were ultracentrifugated 16 h on a gradient of CsCl. 12 fractions were recovered and analysed by Western slot blot to show the fractions containing topoisomerase I (in 1-4 for the untreated control (mock)).

The fractions containing the DNA were identified by measuring absorbance at 260 nm (fractions 8-10). Topoisomerase I was observed only in fractions containing DNA in the presence of inhibitor (CPT or ST1578) or conjugates TFO-L4-10CPT, TFO-ST1578 or LNA-ST1578, suggesting stabilisation of the DNA/topo I cleavage complexes. Upon use of the control TFO (TFO-NH2, TFO-NPh2), topoisomerase I was only present in the first fractions as for the untreated cells (mock)

With this approach, the inventors showed that the conjugates can induce specific breaks by topoisomerase on sites chosen in cell systems. Different topoisomerase I inhibitors can be used and the inhibition will depend on the intrinsic effectiveness of the inhibitor, as the inventors observed with six camptothecin derivatives and indolocarbazole derivatives.

In order to increase the inhibitor effect, chemically modified oligonucleotides can be used, such as for example, PNAs, peptide nucleic acids, 2′OAIkyl ribonucleic acids, oligophosphoramidates, LNAs (RNAs blocked for the conformation of ribose).

The conjugates can be aimed at:
either a single sequence, present, for example, in the human genome for pathologies dependent on the expression of a particular (single) gene, or in viral progenomes (for example, genes responsible for the development of certain viruses, HIV and HSV) or in the genome of parasites. The conjugate then allows the selective inactivation of a gene;
or target sequences common to several genes involved in the maintenance and development of a pathology (for example, oncogenes, growth factors, anti-apoptotic genes, genes controlling the cell cycle and division, which participate in disorders observed in tumorous tumoral cells). The conjugate then allows the simultaneous control of several genes.

In fact, according to the length and sequence of the binding site chosen for the ligand part of the conjugate, the selectivity of the conjugates can be either strict, in order to aim at only a single gene, or loose, in order to target a group of genes.

In the first case, the genome of an integrated virus can be targeted and cleaved specifically by a conjugate directed against a sequence present only in this genome. Within the scope of this application, the inventors extended the approach to include the PPT of the HIV-1 virus.

In the second case, several genes, which are involved in certain tumorous pathologies can be specifically and simultaneously cleaved by topoisomerase I, choosing a common target sequence.

The simultaneous inhibition of genes associated with the acquisition and maintenance of cancerous characteristics makes it possible to target the essential biochemical processes which are specific to the malignant character of the tumorous cells. The inventors chose two groups of genes, involved in the transmission of a growth signal and in the inhibition of apoptosis. In the first case, the growth factor IGF-1 (insulin-like growth factor-1), its receptor IGF-1R and the genes situated downstream in the corresponding signalization cascade were selected. These genes activate cell survival routes and are involved in the proliferation of glioblastomas, hepatocarcinomas and prostate tumours. The inhibition of the IGF-1 or IGF-1R genes by antisense constructions blocks the proliferation of tumours grafted into animals. In the second case, the aim is to induce apoptosis in the cancerous cells, targeting a sequence common to apoptosis-suppressing genes (for example C-IAP1/2, XIAP, survivine, bcl-2, bcl-W, bcl-XL, Mcl-1). Apoptosis or programmed cell death is a controlled fragmentation of the cell executed by caspases. The process is controlled by an equilibrium between the proteins which induce apoptosis and those which inhibit it. The apoptosis-inhibiting genes, by prolonging the life of the cell, increase the probability of genetic events leading to cell malignant transformation; they are often overexpressed in cancerous cells.

To search for sequences capable of forming triple helices common to the group of genes which the inventors wish to target, the latter used the GCG Unix software findpatterns program (Genetics Computer Group, Wisconsin package version 8.1, by Infobiogen;, Villejuif) and also by using the UCSC human genome data base.

A preliminary search for an oligopyrimidine-oligopurine sequence of 12 base pairs (bps) (GGAGGAGGAGGG) common to the IGF-1, IGF-1R and AKT/PKB genes and a 10-bp sequence (GAAGAAGAGG) common to the anti-apoptotic bcl-2, bcl-XL and survivine genes showed the feasibility of the approach. The choice of oligopyrimidineoligopurine sequences is not a limitation of the approach, since these sequences are over-represented in the human genome and the entire gene (regulating regions, coding and non-coding regions) is a potential target for oligonucleotides forming a triple helix. Furthermore oligonucleotide depicted in FIG. 2 recognizes a common sequence present in several genes (Table 1), as for example IGF1R and VEGF involved in the acquisition and maintenance of cancerous characteristics

Moreover, it must not be forgotten that topoisomerase inhibitors have a certain sequence specificity, normally limited to dinucleotides around the cleavage site. In fact, the inventors observed that the binding of the conjugate to the triple helix site is not sufficient to induce strong cleavage and that the presence of a site specific to the inhibitor in the vicinity of the triple helix site is highly preferable for the recruitment and induction of cleavage by topoisomerase. The inventors deduced from this that the targeted sequence should preferably comprise not only the site recognized by the oligonucleotide but also the site of the topoisomerase I inhibitor in its vicinity, thus increasing the selectivity of the conjugate.

Finally, the inventors validated the approach for DNA ligands such as oligonucleotides and polyamides of N-methyl-pyrrole and N-methyl imidazole, but the principle can be extended to other classes of ligands such as zinc finger peptides, for example.

Subjects of the present invention are also:
A method as defined above characterized moreover in that the cleavage by a conjugate (comprising in particular a TFO (triple helix forming oligonucleotide)-topoisomerase inhibitor) is directed to each sequence of said oligopyrimidineoligopurine target gene containing a number of purines of 2-100, preferably 2-30, more effectively with a cleavage site induced by topoisomerase I inhibitor on the 3′ side of the triplex on the oligopyrimidine strand of the target.
A method as defined above characterized moreover in that said cleavage site induced by topoisomerase I inhibitors is positioned 1 to 10 nucleotides from the end of the triple helix.
A method as defined above characterized moreover in that the sequence of said target gene is either a single target sequence present in the human genome on a gene involved in a pathology, or a target present only in a viral or parasitic gene and absent from the human genome, or a sequence present on a group of genes involved in the maintenance or development of a pathology.

The inventors moreover suggested and/or showed that:
Conjugates useful in the method according to the invention should constitute new effective antitumoral agents capable of acting on a group of cell proliferation, growth factor or hormone receptor signalization, and anti-apoptotic genes.
Said minor-groove ligands coupled with a topoisomerase I inhibitor also direct cleavage by the enzyme selectively to the binding site of the ligand and have the same applications as the oligonucleotide-inhibitor conjugates.

### REFERENCES

Arimondo et al., C. R. Acad. Sci. Paris, Series III, Sciences de la Vie/Life Sciences 322. (1999) pp. 785-790.

Arimondo et al., Bioorg. Med. Chem. 8 (2000) pp. 777-784.

Arimondo et al., Bioconj. Chem. 12 (2001) pp. 501-509.

Arimondo et al., Angewandte Chem. Int. Ed. 40 (2001) pp. 3045-3048.

Arimondo et al., J. Biol. Chem. 277 (2002) pp. 3132-3140.

Cantor et al., (1970) Biopolymers 9, pp. 1059-1077.

Grimm et al. (2000) Nucleosides, Nucleotides, Nucleic Acids, pp. 1943-65

Shevelev, A.; Burfeind, P.; Schulze, E.; Rininsland, F.; Johnson, T. R.; Trojan, J.; Chernicky, C. L.; Hélène, C.; Ilan, J. Cancer Gene Ther. 1997, 4, 105-112

Lafarge-Frayssinet, C.; Duc, H. T. Frayssinet, C.; Sarasin, A.; Anthony, D.; Guo, Y.; Trojan, J. Cancer Gene Ther. 1997, 5, 276-285

Hamel Y, Lacoste J, Frayssinet C, Sarasin A, Garestier T, Francois J C, Helene C. Inhibition of gene expression by anti-sense C-5 propyne oligonucleotides detected by a reporter enzyme. Biochem J. 1999 May 1; 339 (Pt 3):547-53.

Petersen M, Wengel J. LNA: a versatile tool for therapeutics and genomics. Trends Biotechnol. 2003 February; 21(2):74-81. Villemin et al. 26(1996) Synthetic Communications pp. 4337-4341.

1. Use of a compound of formula
A-B—C
wherein
A is a DNA sequence-specific ligand capable of simultaneously and specifically recognizing a sequence common to genes of pathological interest;
B is a linker arm, said linker arm being bound to the 3′ end of A;
C is a topoisomerase I poison;
for the preparation of a medicament for the treatment of a disease brought about by the expression of a gene and said gene is inhibited by the stabilized topoisomerase I-mediated DNA cleavage.

2. The use according to claim 1, wherein said genes are genes the expression of which controls the development and maintenance of tumoral state of the cells.

3. The use according to claim 2, wherein said genes are genes selected from the group consisting of IGF-1, IGF-1R, VEGF, BCL2.

4. The use according to claim 1, wherein said gene said genes are genes of an infective microrganism or a virus.

5. The use according to claim 4, wherein said genes are of a HIV or HCV virus.

6. The use according to claim 1, wherein said genes are involved in a dismetabolic disease.

7. The use according to claim 1, wherein said genes are involved in an autoimmune disease.

8. The use according to any one of claim 1, wherein said topoisomerase I poison is selected from the group consisting of camptothecins, rebeccamycins, minor groove ligands and benzimidazoles.

9. The use according to claim 8, wherein said topoisomerase poison is a camptothecin.

10. The use according to claim 9, wherein said camptothecin is selected from the group consisting of, 7-ethyl-10-hydroxycamptothecin and 10-hydroxycamptothecin.

11. The use according to claim 9, wherein said camptothecin is a compound of formula (I) 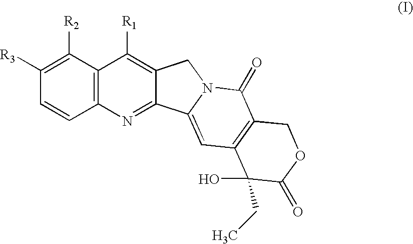
wherein:
R1 is a —C(R5)═N—(O)n—R4 group, in which R4 is hydrogen or a straight or branched C1-C8 alkyl or C2-C8 alkenyl group, or a C3-C10 cycloalkyl group, or a straight or branched (C3-C10) cycloalkyl-(C1-C8) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom selected from an atom of nitrogen, optionally substituted with a (C1-C8) alkyl group, and/or an atom of oxygen and/or of sulphur; said alkyl, alkenyl, cycloalkyl, cycloalkylalkyl, aryl, arylalkyl, heterocyclic or heterocyclo-alkyl groups may optionally be substituted with one or more groups selected from : halogen, hydroxy, keto, C1-C8 alkyl, C1-C8 alkoxy, phenyl, cyano, nitro, —NR6R7, where R6 and R7, which may be the same or different, are hydrogen, straight or branched (C1-C8) alkyl, the —COOH group or one of its pharmaceutically acceptable esters; or the —CONR8R9 group, where R8 and R9, which may be the same or different, are hydrogen, straight or branched (C1-C8) alkyl, phenyl; or R4 is a (C6-C10) aroyl or (C6-C10) arylsulphonyl residue, optionally substituted with one or more groups selected from the group consisting of: halogen, hydroxy, straight or branched C1-C8 alkyl, straight or branched C1-C8 alkoxy, phenyl, cyano, nitro, —NR1OR11, where R10 and R11, which may be the same or different, are hydrogen, straight or branched C1-C8 alkyl; or R4 is a polyaminoalkyl residue; or R4 is a glycosyl residue; R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl; R2 and R3, which may be the same or different, are hydrogen, hydroxyl, straight or branched C1-C8 alkoxy; the N1-oxides, the racemic mixtures, their individual enantiomers, their individual diastereoisomers, their mixtures, and pharmaceutically acceptable salts.

12. The use according to claim 9, wherein said camptothecin is a compound of formula (II) 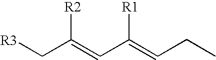
where:
A is saturated or unsaturated straight or branched C1-C8 alkyl, C3-C10 cycloalkyl, straight or branched C3-C10 cycloalkyl-C1-C8 alkyl;
when n and m are equal to 1, then Y is saturated or unsaturated straight or branched C1-C8 alkyl substituted with NR12R13 or N⁺R12R13R14, where R12, R13 and R14, which can be the same or different, are hydrogen or straight or branched C1-C4 alkyl, or Y is BCOOX, where B is a residue of an amino acid, X is H, straight or branched C1-C4 alkyl, benzyl or phenyl, substituted in the available positions with at least one group selected from C1-C4 alkoxy, halogen, nitro, amino, C1-C4 alkyl, or,
if n and m are both 0; Y is 4-trimethylammonium-3-hydroxybutanoyl, both in the form of inner salt and in the form of a salt with an anion of a pharmaceutically acceptable acid, or Y is N⁺R12R13R14, as defined above;
R1 is hydrogen or a —C(R5)═N—(O)p—R4 group, in which p is the number 0 or 1, R4 is hydrogen or a straight or branched C1-C8 alkyl or C1-C8 alkenyl group, or a C3-C10 cycloalkyl group, or a straight or branched (C3-C10) cycloalkyl-(C1-C8) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom selected from an atom of nitrogen, optionally substituted with a (C1-C8) alkyl group, and/or an atom of oxygen and/or of sulphur; said alkyl, alkenyl, cycloalkyl, cycloalkylalkyl, aryl, aryl-alkyl, heterocyclic or heterocyclo-alkyl groups may optionally be substituted with one or more groups selected from: halogen, hydroxy, C1-C8 alkyl, C1-C8 alkoxy, phenyl, cyano, nitro, —NR6R7, where R6 and R7, which may be the same or different, are hydrogen, straight or branched (C1-C8) alkyl, the —COOH group or one of its pharmaceutically acceptable esters; or the —CONR8R9 group, where R8 and R9, which may be the same or different, are hydrogen, straight or branched (C1-C8) alkyl; or R4 is a (C6-C10) aroyl or (C6-C10) arylsulphonyl residue, optionally substituted with one or more groups selected from: halogen, hydroxy, straight or branched C1-C8 alkyl, straight or branched C1-C8 alkoxy, phenyl, cyano, nitro, —NRIOR11, where R10 and R11, which may be the same or different, are hydrogen, straight or branched C1-C8 alkyl; or R4 is a polyaminoalkyl residue; or R4 is a glycosyl residue; R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl; R2 and R3, which may be the same or different, are hydrogen, hydroxyl, straight or branched C1-C8 alkoxy; the N1-oxides, the racemic mixtures, their individual enantiomers, their individual diastereoisomers, their mixtures, and pharmaceutically acceptable salts.

13. The use according to claim 9, wherein said camptothecin is a compound of formula (III) or (IV) 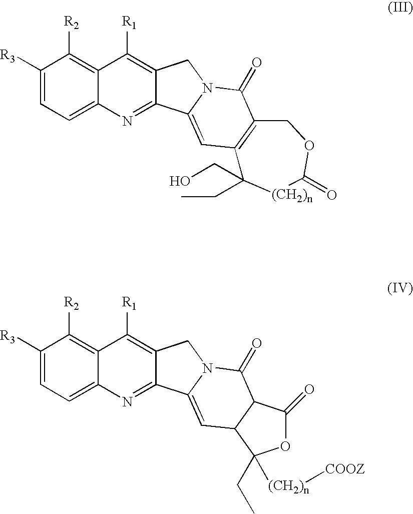
where:
R1 is hydrogen or a —C(R5)═N—(O)p—R4 group, in which p is the integer 0 or 1, R4 is hydrogen or a straight or, branched C1-C8 alkyl or C2-C8 alkenyl group, or a C3-C10 cycloalkyl group, or a straight or branched (C3-C10) cycloalkyl-(C1-C5) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom-selected from an atom of nitrogen, optionally substituted with an (C1-C8) alkyl group, and/or an atom of oxygen and/or of sulphur; said alkyl, alkenyl, cycloalkyl, cycloalkylalkyl, aryl, aryl-alkyl, heterocyclic or heterocyclo-alkyl groups can optionally be substituted with one or more groups selected from the group consisting of: halogen, hydroxy, C1-C8 alkyl, C1-C9 alkoxy, phenyl, cyano, nitro, and —NR6R7, where R6 and R7, which may be the same or different, are hydrogen, straight or branched (C1-C8) alkyl, the —COOH group or one of its pharmaceutically acceptable esters; or the —CONR8R9 group, where R8 and R9, which may be the same or different, are hydrogen, straight or branched (C1-C8) alkyl; or
R4 is a (C6-C10) aroyl or (C6-C10) arylsulphonyl residue, optionally substituted with one or more groups selected from: halogen, hydroxy, straight or branched C1-C8 alkyl, straight or branched C1-C8 alkoxy, phenyl, cyano, nitro, —NR1OR11, where R10 and R11, which may be the same or different, are hydrogen, straight or branched C1-C9 alkyl; or:
R4 is a polyaminoalkyl residue; or
R4 is a glycosyl residue;
R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl;
R2 and R3, which may be the same or different, are hydrogen, hydroxy, straight or branched C1-C8 alkoxy;
n=1 or 2,
Z is selected from hydrogen, straight or branched C1-C4 alkyl; the N1-oxides, the racemic mixtures, their individual enantiomers, their individual diastereoisomers, their mixtures, and their pharmaceutically acceptable salts.

14. The use according to claim 9, wherein said camptothecin is 7-ethyl-10-hydroxycamptothecin or 10-hydroxycamptothecin.

15. The use according to claim 1, wherein said ligand is a triple helix-forming oligonucleotide (TFO).

16. The use according to claim 15, wherein said TFO is is selected from the group consisting of ribonucleic acids, deoxyribonucleic acids, PNAs, peptide nucleic acids, 2′O-alkyl ribonucleic acids, oligophosphoramidates, LNAs.

17. The use according to claim 1, wherein said DNA sequence-specific ligand is a minor groove binder (MGB).

18. The use according to claim 17, wherein said MGB is selected from the group consisting of polyamides of N-methylpyrrole, N-methylimidazole and N-methyl-3-hydroxypyrrole and β-alanine.

19. The use according to claim 1, wherein said linker arm is formed by a succession of carbon atoms and heteroatoms, selected from the group consisting of N or O, of length from 1 to 50, preferably from 2 to 30; and end terminal moieties capable of reacting to give phosphoramide or amide bonds, or thioeters.

20. The use according to claim 19, wherein said linker arm is selected from the group consisting of diamino alkyls and glycols.

21. The use according to claim 1, wherein said medicament is administered by local injection to the site of the disease.

22. The use according to claim 21, wherein said disease is a tumour or an infection.

23. The use according to claim 1, wherein said medicament is administered by systemic route and said compound is vehiculated by a transfection vector, or alone.

24. The use according to claim 20, wherein said transfection vector is selected from the group consisting of nanoparticles, liposomes, cationic lipids and cationic polymers.

25. The use according to claim 1, wherein said medicament is administered by systemic route and in the compound C is selected from the group consisting of 7-(2-aminoethoxyiminomethyl) camptothecin and 7-(3-aminopropoxyiminomethyl) camptothecin.

26. A compound of formula I
A-B—C
wherein
A is a DNA sequence-specific ligand capable of simultaneously and specifically recognizing a sequence common to the genes of pathological interest;
B is a linker arm, said linker arm being bound to the 3′ end of A;
C is a camptothecin derivative of formula I 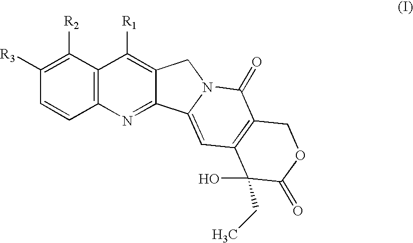
wherein:
R1 is a —C(R5)=N—(0)n—R4 group, in which R4 is hydrogen or a straight or branched C1-C8 alkyl or C2-C8 alkenyl group, or a C3-C10 cycloalkyl group, or a straight or branched (C3-C10) cycloalkyl-(C1-C8) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom selected from an atom of nitrogen, optionally substituted with a (C1-C8) alkyl group, and/or an atom of oxygen and/or of -sulphur; said alkyl, alkenyl, cycloalkyl, cycloalkylalkyl, aryl, arylalkyl, heterocyclic or heterocyclo-alkyl groups may optionally be substituted with one or more groups selected from: halogen, hydroxy, keto, C1-C8 alkyl, C1-C8 alkoxy, phenyl, cyano, nitro, —NR6R7, where R6 and R7, which may be the same or different, are hydrogen, straight or branched (C1-C8) alkyl, the —COOH group or one of its pharmaceutically acceptable esters or the —CONR8R9 group, where R8 and R9, which may be the same or different, are hydrogen, straight or branched (C1-C8) alkyl, phenyl; or R4 is a (C6-C10) aroyl or (C6-C10) arylsulphonyl residue, optionally substituted with one or more groups selected from the group consisting of: halogen, hydroxy, straight or branched C1-C8 alkyl, straight or branched C1-C8 alkoxy, phenyl, cyano, nitro, —NR10R11, where R10 and R11, which may be the same or different, are hydrogen, straight or branched C1-C8 alkyl; or R4 is a polyaminoalkyl residue; or R4 is a glycosyl residue; R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl; R2 and R3, which may be the same or different, are hydrogen, hydroxyl, straight or branched C1-C8 alkoxy; the NI-oxides, the racemic mixtures, their individual enantiomers, their individual diastereoisomers, their mixtures, and pharmaceutically acceptable salts.

27. A compound according to claim 26, wherein R1 is selected from the group consisting of 2-aminoethoxyiminomethyl and 3-aminopropoxyiminomethyl, R₂ and R₃ are hydrogen.

28. A compound of formula I
A-B—C
wherein
A is a DNA sequence-specific ligand capable of simultaneously and specifically recognizing a sequence common to the genes of pathological interest;
B is a linker arm, said linker arm being bound to the 3′ end of A;
C is a camptothecin derivative of formula (II) 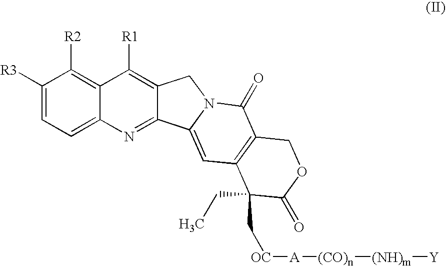
where:
A is saturated or unsaturated straight or branched C1-C8 alkyl, C3-C10 cycloalkyl, straight or branched C3-C10 cycloalkyl-C1-C8 alkyl; when n and m are equal to 1, then Y is saturated or unsaturated straight or branched C1-C8 alkyl substituted with NR12R13 or N⁺R12R13R14, where R12, R13 and R14, which can be the same or different, are hydrogen or straight or branched C1-C4 alkyl, or Y is BCOOX, where B is a residue of an amino acid, X is H, straight or branched C1-C4 alkyl, benzyl or phenyl, substituted in the available positions with at least one group selected from C1-C4 alkoxy, halogen, nitro, amino, C1-C4 alkyl, or,
if n and m are both 0; Y is 4-trimethylammonium-3-hydroxybutanoyl, both in the form of inner salt and in the form of a salt with an anion of a pharmaceutically acceptable acid, or Y is N⁺R12R13R14, as defined above;
R1 is hydrogen or a —C(R5)=N—(0)p—R4 group, in which p is the number 0 or 1, R4 is hydrogen or a straight or branched C1-C8 alkyl or C1-C8 alkenyl group, or a C3-C10 cycloalkyl group, or a straight or branched (C3-C10) cycloalkyl-(C1-C8) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom selected from an atom of nitrogen, optionally substituted with a (C1-C8) alkyl group, and/or an atom of oxygen and/or of sulphur; said alkyl, alkenyl, cycloalkyl, cycloalkylalkyl, aryl, aryl-alkyl, heterocyclic or heterocyclo-alkyl groups may optionally be substituted with one or more groups selected from: halogen, hydroxy, C1-C8 alkyl, C1-C8 alkoxy, phenyl, cyano, nitro, —NR6R7, where R6 and R7, which may be the same or different, are hydrogen, straight or branched (C1-C8) alkyl, the —COOH group or one of its pharmaceutically acceptable esters; or the —CONR8R9 group, where R8 and R9, which may be the same or different, are hydrogen, straight or branched (C1-C8) alkyl; or R4 is a (C6-C10) aroyl or (C6-C10) arylsulphonyl residue, optionally substituted with one or more groups selected from: halogen, hydroxy, straight or branched C1-C8 alkyl, straight or branched C1-C8 alkoxy, phenyl, cyano, nitro, —NR10R11, where R10 and R11, which may be the same or different, are hydrogen, straight or branched C1-C8 alkyl; or R4 is a polyaminoalkyl residue; or R4 is a glycosyl residue; R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl; R2 and R3, which may be the same or different, are hydrogen, hydroxyl, straight or branched C1-C8 alkoxy; the N1-oxides, the racemic mixtures, their individual enantiomers, their individual diastereoisomers, their mixtures, and pharmaceutically acceptable salts.

29. A compound of formula
A-B—C
wherein A is a DNA sequence-specific ligand capable of simultaneously and specifically recognizing a sequence common to the genes of pathological interest;
B is a linker arm, said linker arm being bound to the 3′ end of A;
C is a camptothecin derivative of formula (III) or (IV) 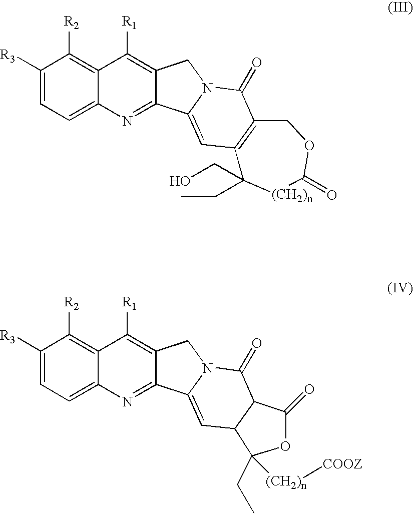
where:
R1 is hydrogen or a —C(R5)═N—(0)p—R4 group, in which p is the integer 0 or 1, R4 is hydrogen or a straight or branched C1-C8 alkyl or C2-C8 alkenyl group, or a C3-C10 cycloalkyl group, or a straight or branched (C3-C10) cycloalkyl-(C1-C5) alkyl group, or a C6-C14 aryl group, or a straight or branched (C6-C14) aryl-(C1-C8) alkyl group, or a heterocyclic group or a straight or branched heterocyclo-(C1-C8) alkyl group, said heterocyclic group containing at least one heteroatom selected from an atom of nitrogen, optionally substituted with an (C1-C8) alkyl group, and/or an atom of oxygen and/or of sulphur; said alkyl, alkenyl, cycloalkyl, cycloalkylalkyl, aryl, aryl- alkyl, heterocyclic or heterocyclo-alkyl groups can optionally be substituted with one or more groups selected from the group consisting of: halogen, hydroxy, C1-C8 alkyl, C1-C9 alkoxy, phenyl, cyano, nitro, and —NR6R7, where R6 and R7, which may be the same or different, are hydrogen, straight or branched (C1-C8) alkyl, the —COOH group or one of its pharmaceutically acceptable esters; or the —CONR8R9 group, where R8 and R9, which may be the same or different, are hydrogen, straight or branched (C1-C8) alkyl; or
R4 is a (C6-C10) aroyl or (C6-C10) arylsulphonyl residue, optionally substituted with one or more groups selected from: halogen, hydroxy, straight or branched C1-C8 alkyl, straight or branched C1-C8 alkoxy, phenyl, cyano, nitro, —NR10R11, where R10 and R11, which may be the same or different, are hydrogen, straight or branched C1-C9 alkyl; or:
R4 is a polyaminoalkyl residue; or
R4 is a glycosyl residue;
R5 is hydrogen, straight or branched C1-C8 alkyl, straight or branched C2-C8 alkenyl, C3-C10 cycloalkyl, straight or branched (C3-C10) cycloalkyl-(C1-C8) alkyl, C6-C14 aryl, straight or branched (C6-C14) aryl-(C1-C8) alkyl;
R2 and R3, which may be the same or different, are hydrogen, hydroxy, straight or branched C1-C8 alkoxy;
n=1 or 2,
Z is selected from hydrogen, straight or branched C1-C4 alkyl; the N1-oxides, the racemic mixtures, their individual enantiomers, their individual diastereoisomers, their mixtures, and their pharmaceutically acceptable salts.

30. A compound of formula
A-B—C
wherein
A is a DNA sequence-specific ligand capable of simultaneously and specifically recognizing a sequence common to the genes of pathological interest;
B is a linker arm, said linker arm being bound to the 3′ end of A;
C is a camptothecin derivative selected from the group consisting of 7-ethyl-10-hydroxycamptothecin and 10-hydroxycamptothecin, succinyl-valyl-20-0-(7-terbutoxyiminomethylcamptothecin) (ST2677), 20S-7-aminoethyliminomethylcamptothecin (ST1578), 20S-7-aminopropyliminomethylcamptothecin (ST2541).

31. A pharmaceutical composition comprising a compound as described in claim 1 in admixture with at least one pharmaceutically acceptable vehicle and/or excipient.

32. The pharmaceutical composition according to claim 31, suitable for injection.

33. The pharmaceutical composition according to claim 31, further comprising a transfection vector.

34. The pharmaceutical composition according to claim 33, wherein said transfection vector is selected from the group consisting of nanoparticles, liposomes, cationic lipids and cationic polymers.

35. An in vitro method for simultaneously inhibiting the expression of several target genes coding for proteins of pathological interest, in particular involved in the development and maintenance of tumors, or viral and pathogenic proteins, or proteins involved in dismetabolic or autoimmune proteins comprising the steps of:
(i) directing the action of at least one topoisomerase I inhibitor towards a site specific to said genes by said conjugate at least one topoisomerase inhibitor to 61 at least one DNA sequence-specific ligand capable of simultaneously and specifically recognizing a sequence common to said target genes,
(ii) recognition by the said ligand of the said conjugate of the said genes in the genome and obtaining the binding of said ligand to said targets,
(iii) induction of topoisomerase I-mediated DNA cleavage, and inhibiting the expression of the said genes.

36. The method according to claim 35, wherein the sequences of said target genes include the site of the topoisomerase inhibitor in their vicinity.

37. The method according to claim 35, wherein said at least one topoisomerase inhibitor is chosen from the group comprising intercalating agents, such as indolocarbazoles and derivatives thereof, non-intercalating agents, such as camptothecin and derivatives thereof, minor-groove ligands, such as benzimidazoles and derivatives thereof.

38. The method according to claim 35, wherein said at least one ligand is selected from the group consisting of ribonucleic acids, deoxyribonucleic acids, PNAs, peptide nucleic acids, 2′O-alkyl ribonucleic acids, oligophosphoramidates, LNAs, and correspond to TFO when it forms a triple helix and MGB when it binds to the minor groove, and is then chosen from polyamides of N-methylpyrrole, N-methylimidazole and N-methyl-3-hydroxypyrrole and β-alanine.

39. The method according to claim 35, wherein the cleavage by a conjugate comprising a triple helix forming oligonucleotide-topoisomerase inhibitor is directed to each oligopyrimidineoligopurine sequence of said target genes containing a number of purines between 2 and 100, preferably 10-30 with a cleavage site induced by the topoisomerase I inhibitor on the 3′ side of the triplex on the oligopyrimidine strand of the target.

40. The method according to claim 39, wherein said cleavage site induced by the topoisomerase inhibitor is positioned 3 to 8 nucleotides from the end of the triple helix.

41. The method according to claim 35, wherein the sequences of said target genes are present in a group of genes, in particular genes involved in the transmission of an apoptosis growth and/or inhibition signal.


SEQUENCE LISTING (23 sequences)

SEQ ID NO: 1 - DNA, 16 bp, artificial sequence
  PPT HIV-1 virus
aaaagaaaagggggga

SEQ ID NO: 2 - DNA, 16 bp, artificial sequence
  PPT HIV-1 virus
ttttcttttcccccct

SEQ ID NO: 3 - DNA, 22 bp, artificial sequence
  PPT HIV-1 virus
gaagagggagagagagagaagg

SEQ ID NO: 4 - DNA, 23 bp, artificial sequence
  PPT HIV-1 virus
tcttctccctctctctctcttcc

SEQ ID NO: 5 - RNA, 16 nt, artificial sequence
  Oligonucleotide
cucucucucuuuuuuu

SEQ ID NO: 6 - RNA, 18 nt, artificial sequence
  Oligonucleotide
cucucucucuuuuuuucu

SEQ ID NO: 7 - RNA, 20 nt, artificial sequence
  Oligonucleotide
cucucucucuuuuuuucucu

SEQ ID NO: 8 - RNA, 23 nt, artificial sequence
  triplex-forming oligonucleotide
cucucucucuuuuuuucucuucu

SEQ ID NO: 9 - DNA, 61 bp, artificial sequence
  Oligonucleotide
aattcaagcttacactccctatcagtgatagagagagagaaaaaaagagaagatctgagct

SEQ ID NO: 10 - DNA, 59 bp, artificial sequence
  Oligonucleotide
aagttcgaatgtgagggatagtcactatctctctctctttttttctcttctagactcga

SEQ ID NO: 11 - DNA, 15 bp, artificial sequence
  Region of the pGL promotor
gagaagatctgagct

SEQ ID NO: 12 - protein, 23 aa, artificial sequence
  Region of the pGL3 promotor
KTGAFLLQGFIQDRAGRMAGETP

SEQ ID NO: 13 - DNA, 16 bp, artificial sequence
  Oligonucleotide
gaaggagagaaaaaaa

SEQ ID NO: 14 - DNA, 16 bp, artificial sequence
  Oligonucleotide
cttcctctcttttttt

SEQ ID NO: 15 - DNA, 16 bp, artificial sequence
  Oligonucleotide
gagactcagaaaaaaa

SEQ ID NO: 16 - DNA, 16 bp, artificial sequence
  Oligonucleotide
ctctgagtcttttttt

SEQ ID NO: 17 - DNA, 76 bp, artificial sequence
  Oligonucleotide
gaattcaagcttacactccctatcagtgatagagagagagaaaaaagagaagatctgagctcggtaccctaggatc

SEQ ID NO: 18 - DNA, 77 bp, artificial sequence
  Oligonucleotide
cttaagttcgaatgtgagggatagtcactatctctctctctttttttctcttctagactcgagccatgggatcctag

SEQ ID NO: 19 - RNA, 16 nt, artificial sequence
  Oligonucleotide
cucucucucuuuuuuu

SEQ ID NO: 20 - RNA, 16 nt, artificial sequence
  Oligonucleotide
cucucucucuuuuuuu

SEQ ID NO: 21 - RNA, 16 nt, artificial sequence
  Oligonucleotide
cucucucucuuuuuuu

SEQ ID NO: 22 - RNA, 16 nt, artificial sequence
  Oligonucleotide
uuuucuuuuccccccu

SEQ ID NO: 23 - RNA, 15 nt, artificial sequence
  Oligonucleotide
uuucuuuuccccccu